(19) United States
(12) Patent Application Publication
(10) Pub. No.: US 2007/0078177 A1
(43) Pub. Date: Apr. 5, 2007

(54) METHODS AND COMPOSITIONS FOR TREATING NON AGE RELATED HEARING IMPAIRMENT IN A SUBJECT

(75) Inventor: Jianxin Bao, St. Louis, MO (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: WASHINGTON UNIVERSITY IN ST. LOUIS, St. Louis, MO (US)

(21) Appl. No.: 11/537,149

(22) Filed: Sep. 29, 2006

Related U.S. Application Data
(60) Provisional application No. 60/722,330, filed on Sep. 30, 2005.

(51) Int. Cl. A61K 31/4015

(52) U.S. Cl. 514/425

(57) ABSTRACT

The invention provides methods for treating non-age related hearing impairments, especially sensorineural and neural hearing loss, in a subject in need of such treatment.

### CROSS-REFERENCE TO RELATED APPLICATION

This application claims priority from Provisional Application Ser. No. 60/722,330 filed on Sep. 30, 2005, which is hereby incorporated by reference in its entirety.

### FIELD OF THE INVENTION

The present invention generally relates to treating non-age related hearing impairment, especially sensorineural and neural hearing loss, in a subject in need of such treatment. More specifically, the present invention provides methods of using a therapeutic agent for the treatment of non-age related hearing loss due to noise, surgical procedures, toxins, or other stressors.

### BACKGROUND OF THE INVENTION

In the United States, almost thirty million people suffer from some degree of hearing loss or deafness and the condition costs the nation more than 50 billion dollars each year, more than epilepsy, multiple sclerosis, spinal injury, stroke, Huntington's, and Parkinson's disease combined. There are three types of hearing loss, namely conductive hearing loss, sensorineural hearing loss, and mixed hearing loss, a combination of conductive and sensorineural hearing loss. Conductive hearing loss results from impairment of the external or middle ear, which is commonly mechanical in nature, i.e., impacted earwax, presence of a foreign body, ear infection (otitis media, external otitis), and thus can be corrected by medicine and/or surgery. Sensorineural hearing loss includes sensory hearing loss, which is due to disorders in the cochlea, and neural hearing loss, which results from damage to or the absence of the vestibulocochlear nerve, also referred to as cranial nerve VIII or the auditory nerve. The vast majority of cases of hearing loss are sensorineural and are caused by a loss of hair cells in the cochlea (e.g., there are some 16,000 hair cells in the cochlea). Each hair cell is characterized by a bundle of stereocilia that projects from its apical surface. These stereocilia enable hair cells to tranduce mechanical stimuli derived from sound into neural (electrical) signals that cranial nerve VIII then transmits to the brain. Because these cells are not replaced by cellular division, their disappearance leads to a gradual loss of the sense of hearing.

Sensorineural hearing loss may be due to a genetic disorder or caused by any one of a number of events, including ototoxic drug exposure, sound trauma, surgical trauma (i.e., related to the surgical removal of a tumor on cranial nerve VIII), physical trauma (i.e., due to a fracture of the temporal bone affecting the inner and middle ear or due to a shearing injury affecting cranial nerve VIII), exposure to mercury, lead, or toluene, disease, and infection, such as meningitis. One disease that causes sensorineural hearing loss is Meniere's disease, which afflicts approximately 0.1-0.5% of the adult population. The disease is typically characterized by episodes of vertigo, hearing loss, and tinnitus, lasting for hours up to a few days. During the intermittent time periods, an individual may suffer from tinnitus and hearing loss. Usually Meniere's disease is unilateral, affecting only one ear, but in some 12% of cases, both ears are affected (bilateral). Complete deafness in an affected ear has been reported to occur at a rate of about 10%. In spite of the fact that Meniere's disease is relatively common and disabling, there is no causal therapy for the disease. Thus, certain therapies focus on treating the symptoms of the disease, i.e., hearing loss and tinnitus.

Tinnitus, the perception of sound in the absence of acoustic stimulus (i.e., ringing, roaring, whooshing), is often associated with sensorineural hearing loss. More than 50 million Americans suffer from some form or degree of tinnitus. Some 12 million of these seek medical attention, and about two million patients are so severely incapacitated that they cannot perform everyday tasks. The pathophysiologic etiology of tinnitus is not well understood. The causes of tinnitus are similar to the causes of hearing loss, e.g., acoustic trauma, ototoxic drugs, and infections. As noted above, tinnitus is also a symptom of Meniere's disease. There are many variants of tinnitus, some of which are caused by disorders in the external ear and can be successfully treated. However, much like sensorineural hearing loss, tinnitus is most commonly associated with the inner ear and it is very difficult to treat.

Currently, there are no clinically proven medications for the treatment of hearing loss (sensorineural and neural) or tinnitus associated with the inner ear, and a medication that could be used to prevent, alleviate or eliminate hearing loss (or tinnitus) would thus be very desirable. The most common remedy for individuals suffering from severe sensorineural hearing loss is a hearing aid, which functions to amplify sound. Hearing aids are non-invasive and can improve an individual's ability to hear. However, hearing aids can often be quite conspicuous and embarrassing to the wearer and hearing aids do not return hearing to normal levels. Furthermore, hearing aids amplify sound indiscriminately, sometimes amplifying sounds that an individual does not wish to hear, such as environmental noise. And, while a hearing aid amplifies sound, it doesn't necessarily improve the clarity of the sound. Finally, hearing aids, especially smaller, less conspicuous hearing aids, are expensive.

A far less common alternative to a hearing aid is an electronic cochlear implant. In most cases of severe to profound sensorineural hearing loss, while there has been a significant loss of hair cells in the cochlea, the cochlear nerves, i.e., cranial nerve VIII, which carry neural (electrical) signals to the brain, are still intact. An electronic cochlear implant bypasses hair cells and stimulates cochlear nerves directly, using electrical impulses. After a successful cochlear implantation, patients can recognize environmental sound, understand spoken language, and listen to music. However, a cochlear implant requires invasive surgery, preceded by rigorous testing of the individual to determine whether she is a candidate for the surgery. Other limitations to this technology include the failure of individual electrodes within the ear, requiring programming with a more limited number of electrodes. There is also a small percentage of total device failures, which require complete surgical replacement of the device. With some implant models, the internal device must be removed completely or in part if the patient ever needs MRI scanning. Further, the inner ear electrodes are placed in close proximity to the facial nerve, and, in some patients, the desired level of electrical stimulation cannot be achieved without stimulating the facial nerve and causing facial twitching. And, in a small minority of patients the device simply fails to provide much sound information to the brain; a few patients, while able to hear environmental sounds, struggle to understand speech or other more complex sounds. Most importantly, even in patients with the most ideal results, cochlear implantation never provides hearing that approaches the frequency specificity and sophistication of normal hearing.

Due to the myriad of disadvantages associated with expensive, low-fidelity, battery-powered hearing aids and the invasive nature and other disadvantages of cochlear implantation, methods that treat sensorineural hearing loss and enable people to regain their hearing are needed. Furthermore, hearing aids and cochlear implants are not a remedy for neural hearing loss, because, in neural hearing loss, the auditory nerve is not able to pass on enough sound information to the brain (an auditory brainstem implant (ABI) may help in some cases). But, there remains a need for a therapeutic agent capable of treating sensorineural hearing loss, as well as neural hearing loss and tinnitus, which is commonly associated with sensorineural hearing loss.

### SUMMARY OF THE INVENTION

Among the several aspects of the invention is provided a method for treating a non-age related hearing impairment in a subject in need of such treatment. In one embodiment, the method comprises administering a derivative of a succinimide compound to the subject.

Another aspect of the invention provides a method for treating a non-age related hearing impairment in a subject in need of such treatment, the method comprising administering a derivative of an oxazolidiendione compound to the subject.

A further aspect of the invention provides a method for treating damage to sensory hair cells or damage to cochlear neurons in a subject in need of such treatment, the method comprising administering a derivative of a succinimide compound to the subject.

An additional aspect of the invention provides a method for treating damage to sensory hair cells or damage to cochlear neurons in a subject in need of such treatment, the method comprising administering a derivative of an oxazolidiendione compound to the subject.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

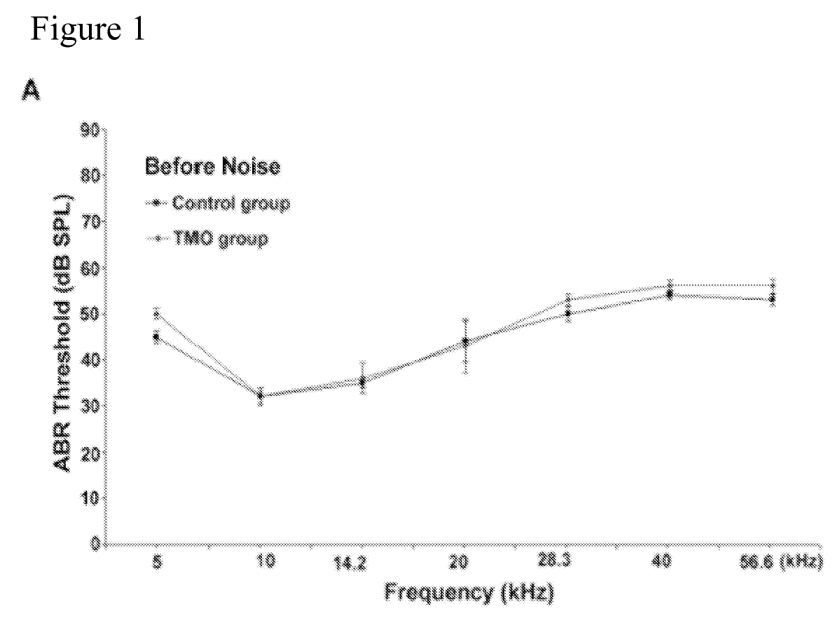

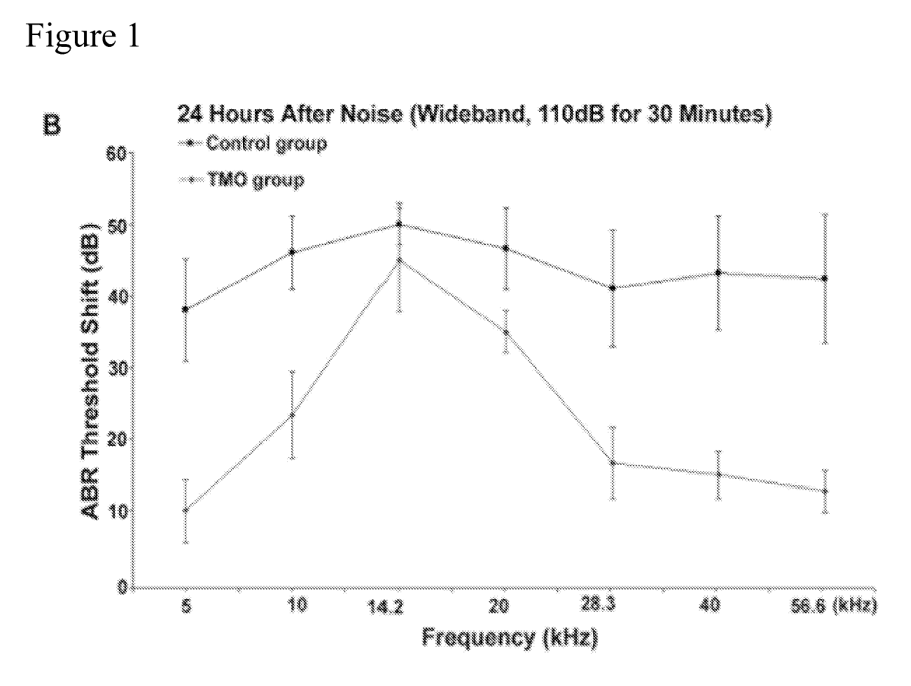

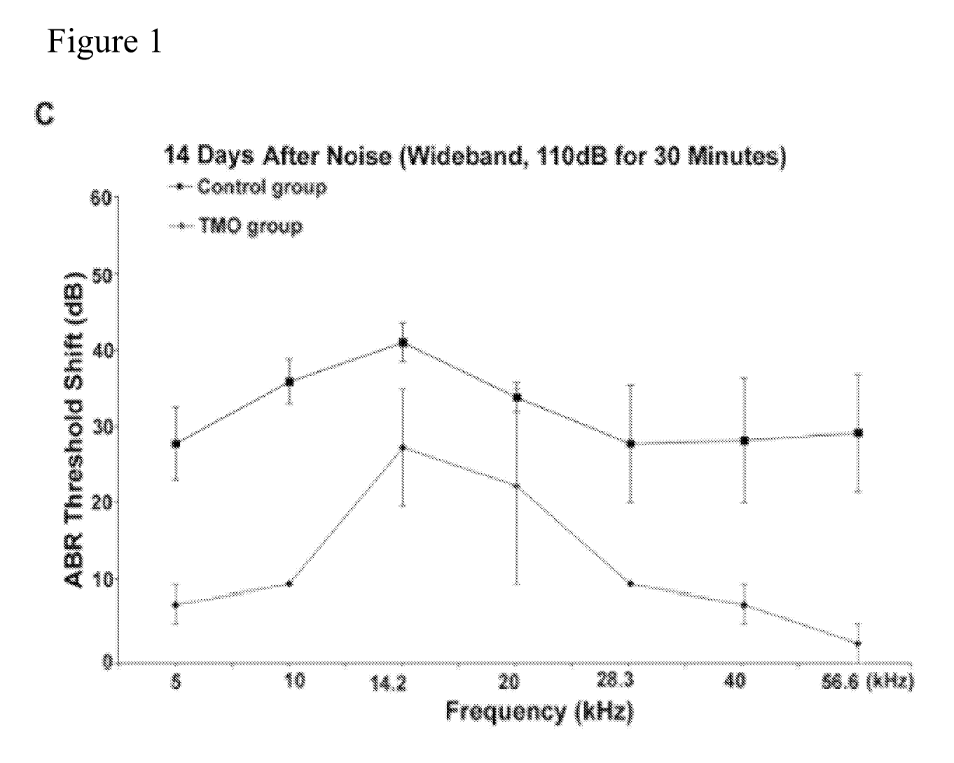

FIG. 1 depicts a series of images illustrating the prophylactic function of trimethadione (TMO) against noise-induced hearing loss. Inbred mice were fed TMO at 200 mg/kg body weight/day in their drinking water for three weeks prior to noise exposure. Body weight and water uptake were monitored every three days. TMO solution was maintained in colored bottles and changed every three days. To examine the effects of TMO on noise-induced hearing loss, auditory brainstem recording (ABR) functional analysis was performed to measure hearing thresholds in mice before and after noise exposure. Before noise exposure and one week after TMO treatment, there was no difference in ABR thresholds between control mice and TMO-treated mice (FIG. 1A). Twenty-four hours after the noise exposure (wideband, 110 dB for 30 minutes), the temporary threshold shift (TTS) was significantly lower at all seven frequencies tested in the TMO-treated mice compared to the control mice (FIG. 1B). Fourteen days after the noise exposure, the permanent threshold shift (PTS) was also significantly lower at all seven frequencies tested in the TMO-treated mice compared to the control mice (FIG. 1C).

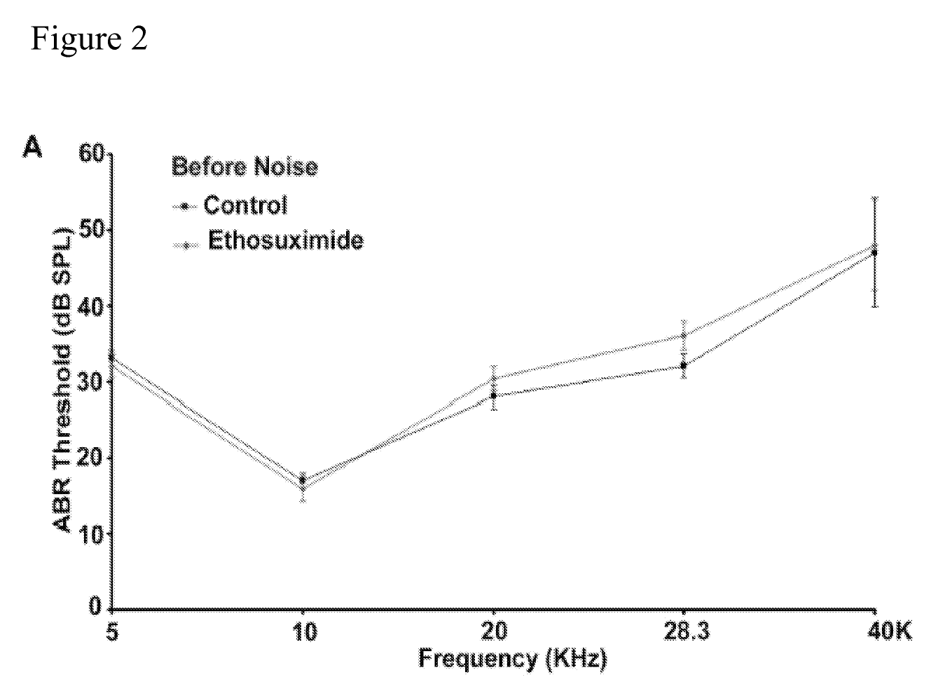

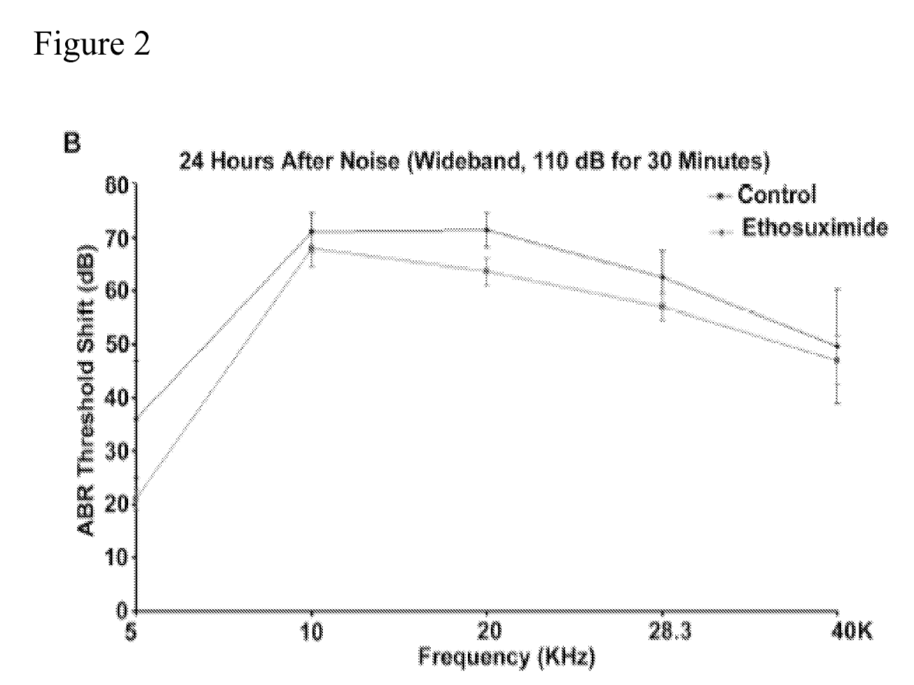

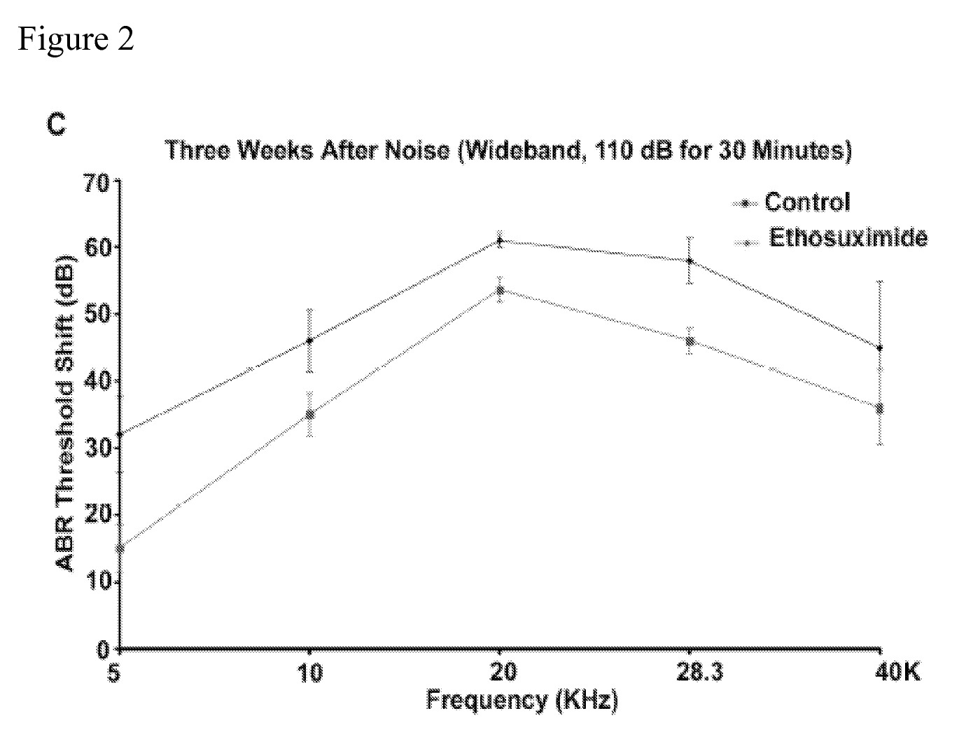

FIG. 2 depicts a series of images illustrating the prophylactic function of ethosuximide against noise-induced hearing loss. Inbred mice were fed ethosuximide at 400 mg/kg body weight/day in their drinking water for three weeks, beginning immediately after the noise exposure (wideband, 110 dB for 30 minutes). To examine the effects of ethosuximide on noise-induced hearing loss, ABR functional analysis was performed to measure hearing thresholds in mice before and after noise exposure. Before noise exposure, there was no difference in ABR thresholds between control mice and ethosuximide-treated mice (FIG. 2A). Twenty-four hours after the noise exposure, the temporary threshold shift (TTS) was significantly lower at all seven frequencies tested in the ethosuximide-treated mice compared to the control mice (FIG. 2B). Twenty-one days after the noise exposure, the permanent threshold shift (PTS) was also significantly lower at all seven frequencies tested in the ethosuximide-treated mice compared to the control mice (FIG. 2C).

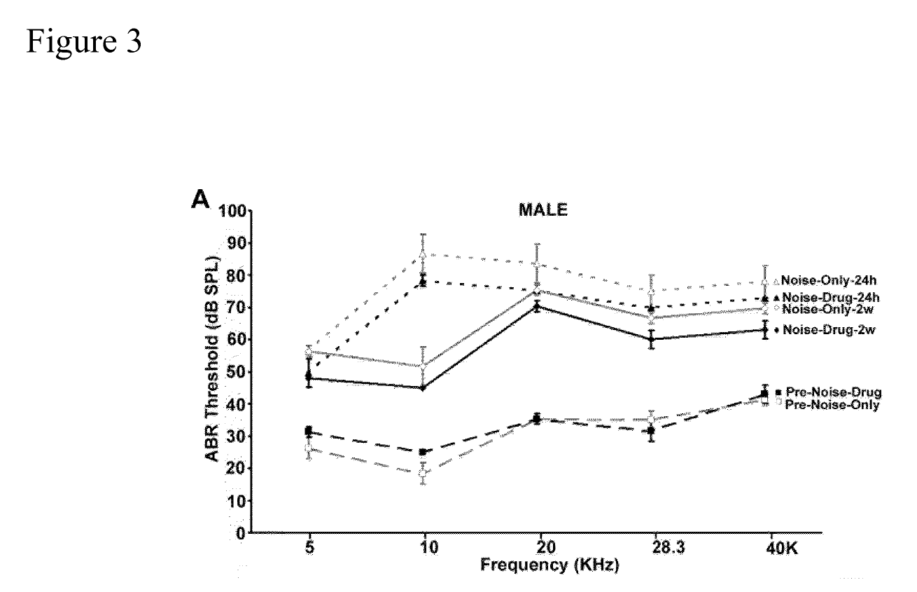

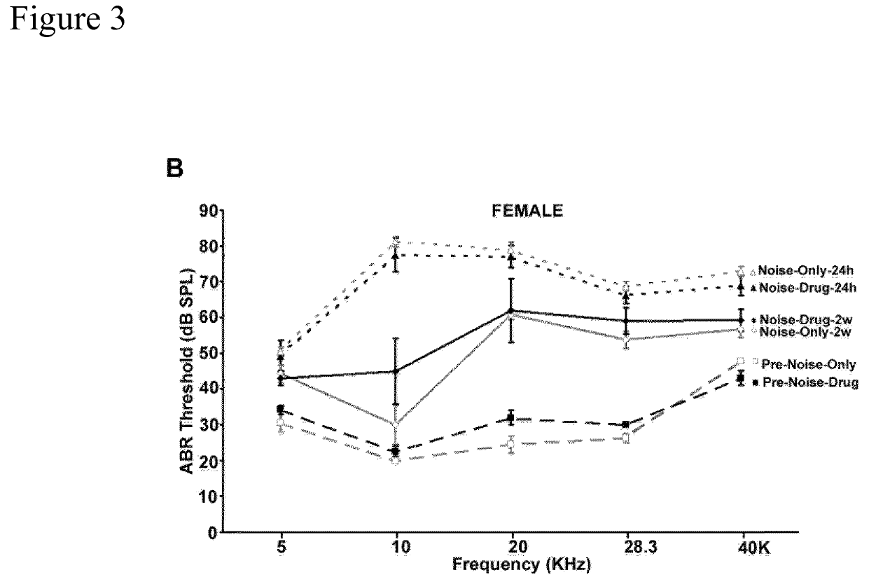

FIG. 3 depicts graphs of the therapeutic function of trimethadione on noise induced hearing loss (NIHL). (A) ABR thresholds for male mice (n=3 for each group) at 3 months old. A significant protection from NIHL for both TTS and PTS was found in male mice, which received trimethadione in their drinking water right after the noise exposure (Mean+/−S.D; one-way ANOVA, p<0.05). (B) ABR threshold for female mice (n=4 for each group) at 3 months old (Mean+/−S.D.).

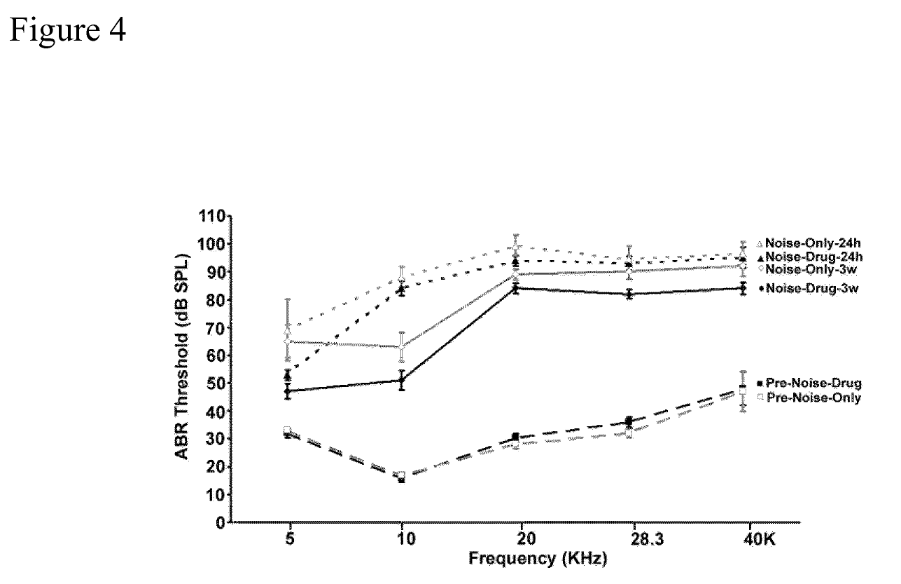

FIG. 4 depicts a graph of the therapeutic function of ethosuximide on NIHL. (A) ABR thresholds for male (n=5 for each group) mice at 3 months old. A significant protection from NIHL for both TTS and PTS was found in mice, which received ethosuximide in their drinking water right after the noise exposure (Mean+/−S.D.; one-way ANOVA, p<0.05).

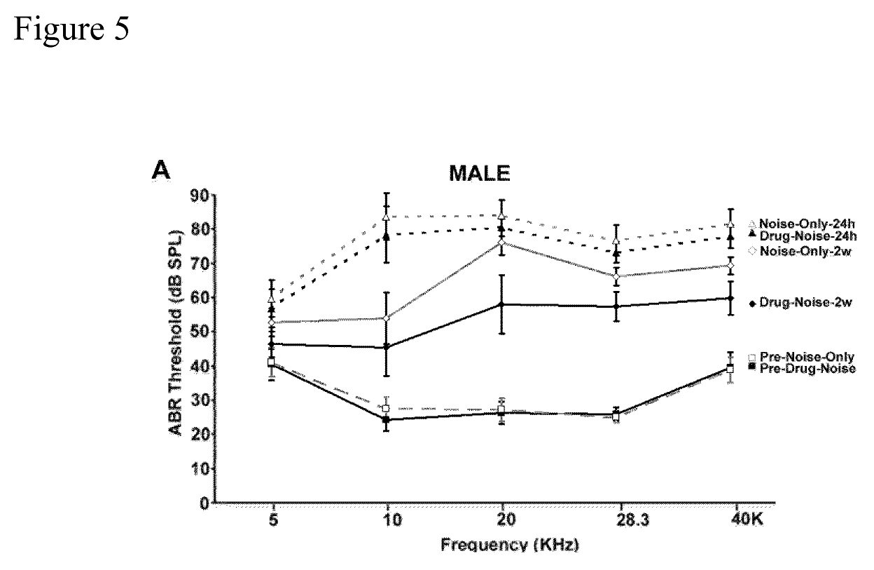

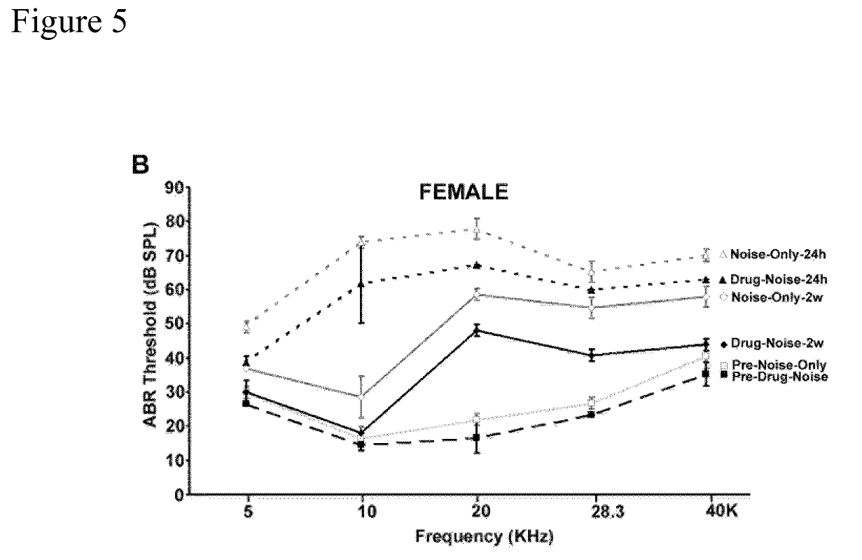

FIG. 5 depicts graphs of the prophylactic function of trimethadione. (A) ABR thresholds for male mice at 3 months old (n=6 for the Noise-Only group, and n=7 for the Drug-Noise group; Mean+/−S.D.). (B) ABR threshold for female mice at 3 months old (n=3 for each group). A significant protection from NIHL for both TTS and PTS was found in both male and female mice, which received trimethadione in their drinking water two weeks before the noise exposure (Mean+/−S.D.; one-way ANOVA, p<0.05).

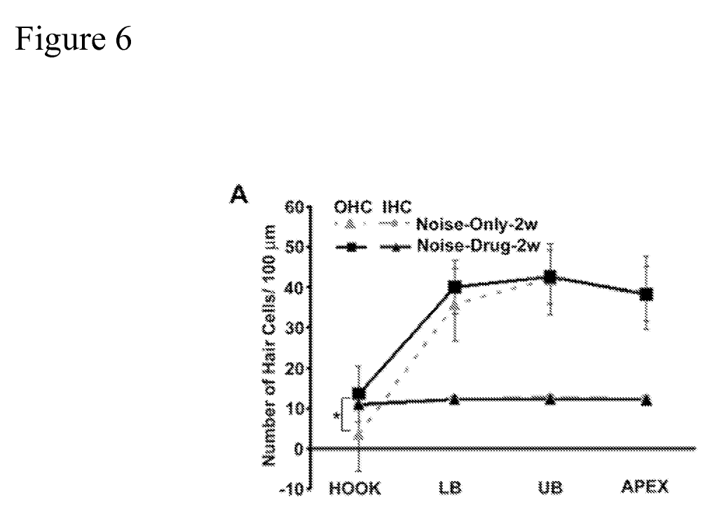

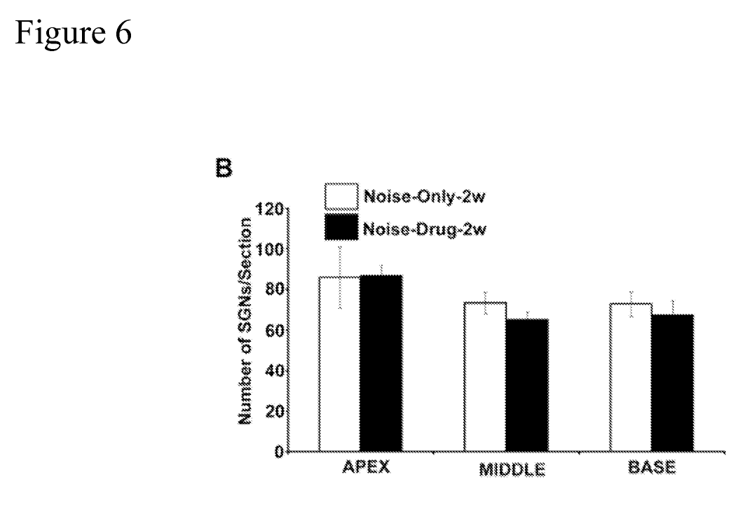

FIG. 6 depicts graphs showing quantitative comparisons of outer hair cells (OHCs), inner hair cells (IHCs), and spiral ganglion neurons (SGNs) between the control and trimethadione-treated mice. (A) Counting of IHC and OHC at the hook, low base (LB), upper base (UB), and apex region of the cochlea at 100 μm interval from 3-months-old male mice (n=3 for each group). There was no significant difference for both OHC and IHC numbers between the control and trimethadione-treated mice, although there was a trend for fewer OHC close to the base in the control mice (Mean+/−S.D.). (B). Counting of SGNs at the apex (including both sides), the right side of SGNs at the middle and base regions from the same male mice. The density of SGNs was not significantly different between the control and the trimethadione-treated mice (Mean+/−S.D.).

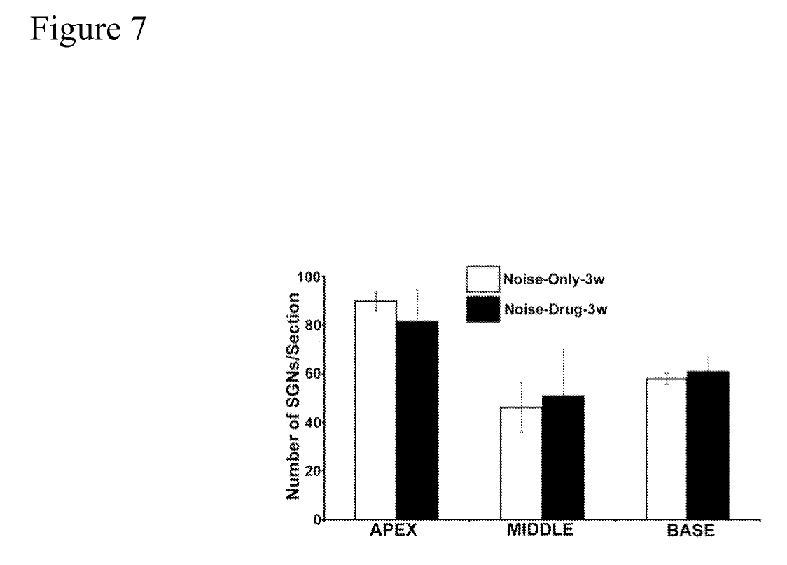

FIG. 7 depicts a graphs of a quantitative comparison of SGN numbers between the control and ethosuximide-treated mice. Counting of SGNs at the apex (including both sides), the right side of SGNs at the middle and base regions from male mice at 3 months (n=4 for each group). The density of SGNs was not significantly different between the control and the ethosuximide-treated mice (Mean+/−S.D.).

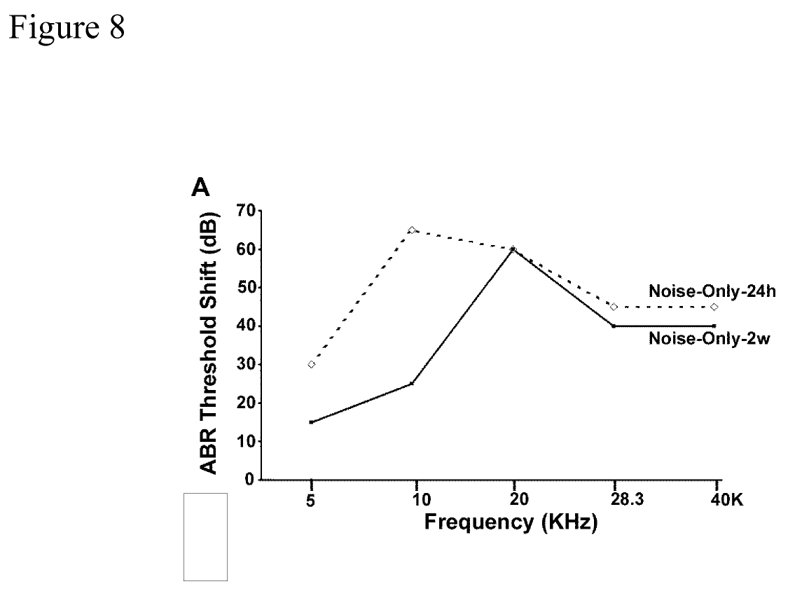

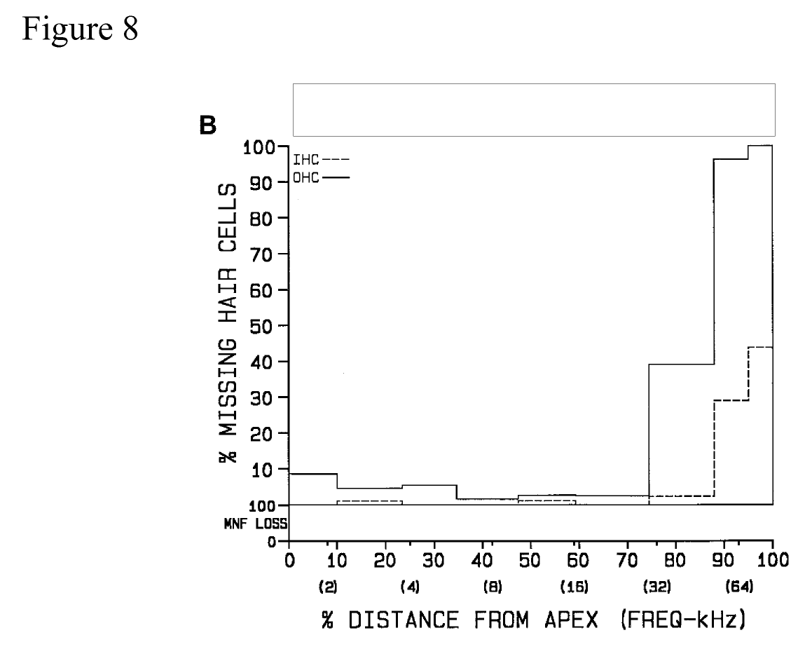

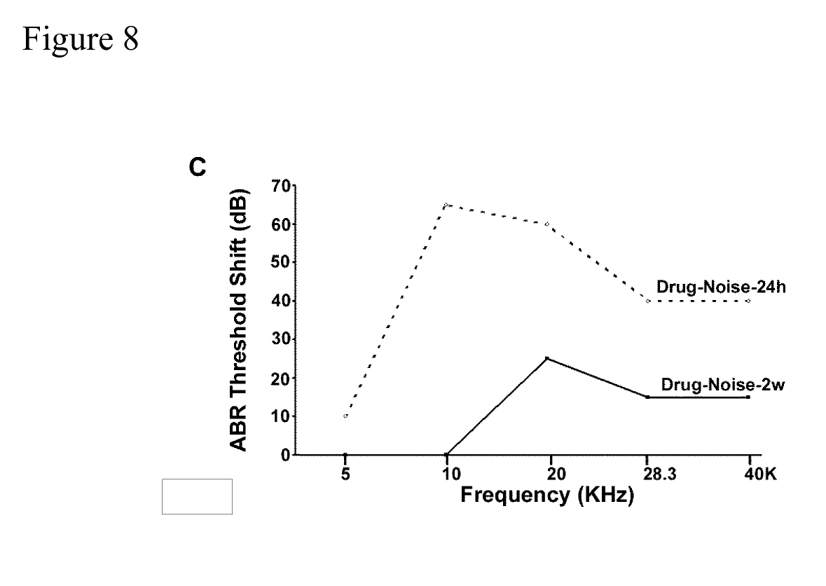

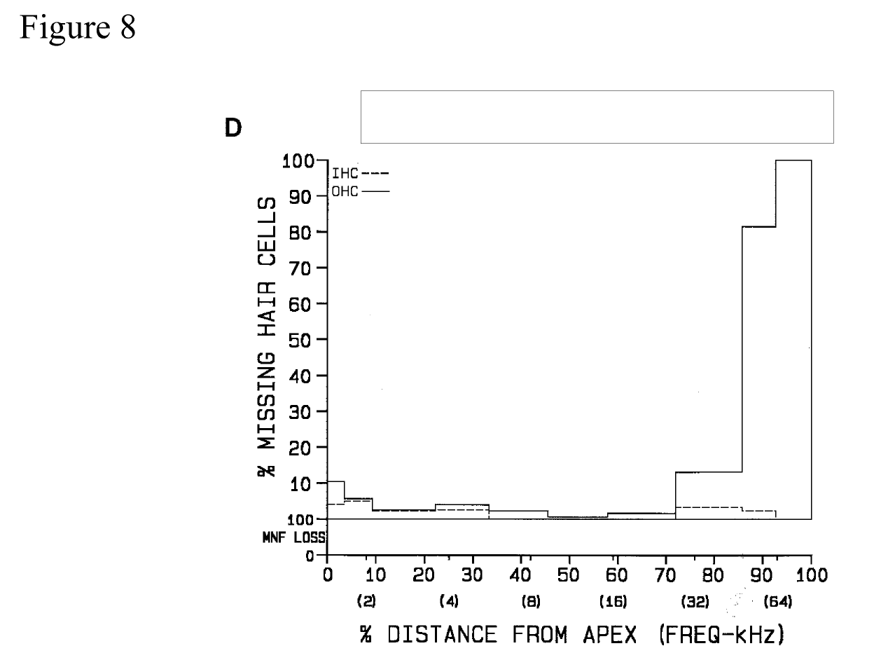

FIG. 8 depicts graphs of the ABR threshold shifts and cytocochleogram for a pair of mice testing the prophylactic function of trimethadione. (A) and (B) ABR threshold shifts and cytocochleogram for the control mouse (male, 3 months old). (C) and (D) ABR threshold shifts and cytocochleogram for the trimethadione-treated mouse (male, 3 months old). Both of the mice were exposed to the noise in the same cage at the same time.

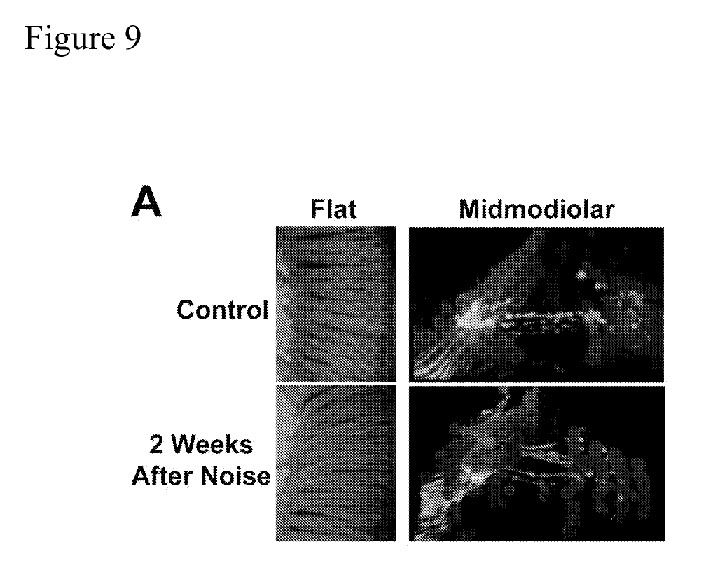

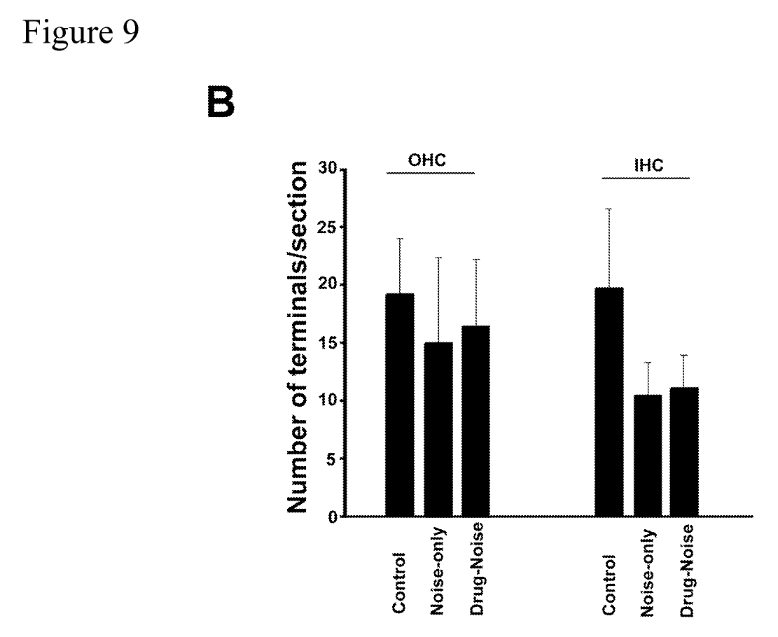

FIG. 9 illustrates changes of YFP positive SGN afferent terminals in Y12 mice. (A) YFP positive terminals in the cochlea of Y12 mice. Left panel, SGN afferent fibers in the whole mount (top, without noise; bottom, 2 weeks after noise). Right panel, 3-D reconstruction of 1 mm images for two midmodiolar sections at 40 mm (top, without noise, bottom, 2 weeks after noise), in which SGN afferent terminals are found underneath IHCs (red, by immunostaining for calretinin), and both afferent and efferent terminals are found underneath OHCs. (B) Quantification of YFP positive terminals underneath IHCs and OHCs among the Control, Noise-Only, and Drug-Noise groups (n=3 for each group, Mean+/−S.D.)

### DETAILED DESCRIPTION OF THE PREFERRED EMBODIMENTS

The present invention provides methods for treating non-age related impairment, including hearing loss, especially sensorineural and neural hearing loss, in a subject in need of such treatment. More specifically, the present invention provides a method of using a therapeutic agent for the treatment of hearing loss due to noise, surgical procedures, toxins, or other stressors.

I. Therapeutic Agents

It has been discovered that the administration of a therapeutically effective amount of a derivative of a succinimide, a derivative of an oxazolidiendione, zonisamide, or flunarizine provides a method for treating non-age related hearing impairment in a subject in need of such treatment. A number of suitable such compounds or isomers, pharmaceutically acceptable salts, esters, or prodrugs thereof may be employed in the methods of the present invention. Succinimide corresponds to Formula A-1, oxazolidiendione corresponds to Formula B-1, zonisamide corresponds to Formula C-1, and flunarizine corresponds to Formula D-1.

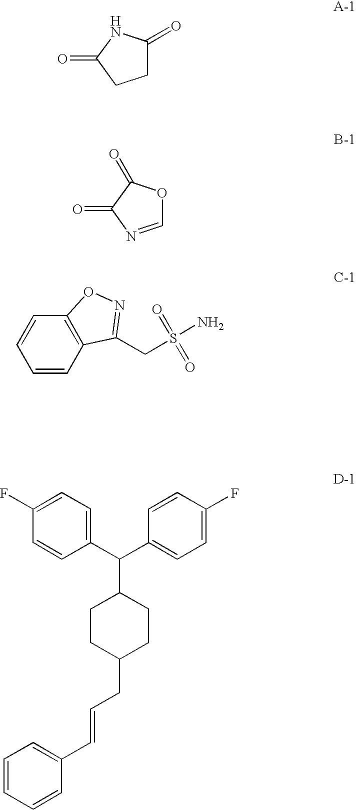

In such embodiments, the compound may be a derivative of a succinimide compound, for example, ethosuximide, Formula A-2 or an isomer, a pharmaceutically acceptable salt, ester, or prodrug of a compound having Formula A-2.

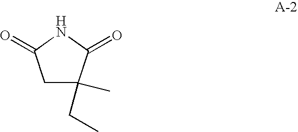

In other embodiments, the compound may be a succinimide derivative selected from the consisting of methsuximide, Formula A-3, phensuximide, Formula A-4, α-methyl-α-phenylsuccinimide, Formula-A-5, tetramethylsucinimide, Formula A-6 or an isomer, a pharmaceutically acceptable salt, ester, or prodrug of a compound having Formula A-3, Formula A-4, Formula A-5 or Formula A-6.

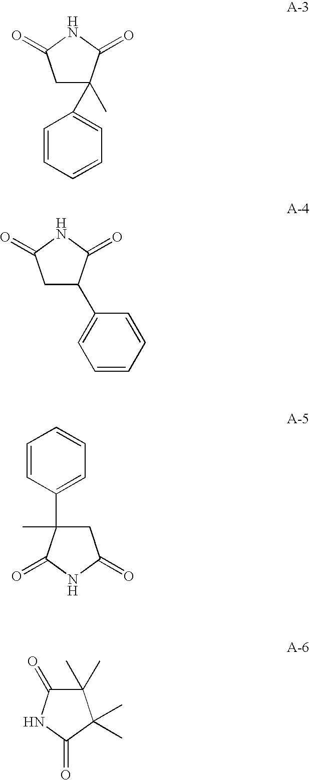

In further embodiments, the compound may be a derivative of an oxazolidiendione compound, for example, trimethadione, Formula B-2 or an isomer, a pharmaceutically acceptable salt, ester, or prodrug of a compound having Formula B-2.

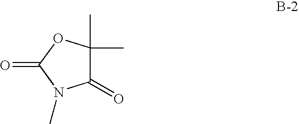

In yet further embodiments, the compound may be the oxazolidiendione derivative paramethadione, Formula B-3 or an isomer, a pharmaceutically acceptable salt, ester, or prodrug of a compound having Formula B-3.

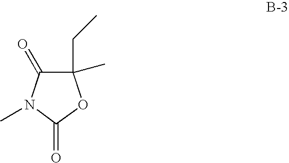

In another embodiment, the compound may be zonisamide, Formula C-1 or an isomer, a pharmaceutically acceptable salt, ester, or prodrug of a compound having Formula C-1.

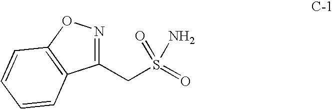

In still another embodiment, the compound may be flunarizine, Formula D-1 or an isomer, a pharmaceutically acceptable salt, ester, or prodrug of a compound having Formula D-1.

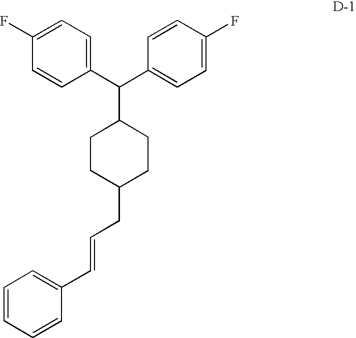

The therapeutic agents employed in the present invention can exist in tautomeric, geometric or stereoisomeric forms. The present invention contemplates all such compounds, including cis- and trans-geometric isomers, E- and Z-geometric isomers, R- and S-enantiomers, diastereomers, d-isomers, I-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures of R and S forms for each stereocenter present.

The therapeutic agents utilized in the present invention may be in the form of free bases or pharmaceutically acceptable acid addition salts thereof. The term “pharmaceutically-acceptable salts” embraces salts commonly used to form alkali metal salts and to form addition salts of free acids or free bases. The nature of the salt may vary, provided that it is pharmaceutically acceptable. Suitable pharmaceutically acceptable acid addition salts of compounds of use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

As will be appreciated by the skilled artisan, the therapeutic agents of the present invention can be formulated into pharmaceutical compositions and administered by a number of different means that will deliver a therapeutically effective dose. They can be administered locally or systemically. Such compositions can be administered orally, parenterally, by inhalation spray, intrapulmonary, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intraarterial, intraperitoneal, intracochlear, or intrasternal injection, or infusion techniques. The therapeutic agents of the present invention can be administered by daily subcutaneous injection or by implants. The agents can be administered in liquid drops to the ear canal, delivered to the scala tympani chamber of the inner ear, or provided as a diffusible member of a cochlear hearing implant. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols may be used. Mixtures of solvents and wetting agents such as those discussed above are also useful.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of a therapeutic agent of the invention will vary depending upon the patient and the particular mode of administration. In general, the pharmaceutical compositions may contain a therapeutic agent in the range of about 1 to 1000 mg, more typically, in the range of about 10 to 800 mg and still more typically, between about 50 and 600 mg. A daily dose of about 1 to 4000 mg, or more typically, between about 10 and 3000 mg, and even more typically, from about 100 to 2500 mg, may be appropriate. The daily dose depends on various factors, particularly the age and body weight of the subject. A reduced dosage is generally administered to children. The daily dose is generally administered in one to about four doses per day.

In certain embodiments, when the therapeutic agent comprises ethosuximide, it is typical that the amount used is within a range of from about 100 to about 2000 mg/day, and even more typically, from about 300 to about 1800 mg/day. For children up to six years of age, it is typical that the amount used is within a range of from about 50 to about 1500 mg/day, and even more typically, from about 150 to about 1200 mg/day.

In other embodiments, when the therapeutic agent comprises methsuximide, it is typical that the amount used is within a range of from about 50 to about 1800 mg/day, and even more typically, from about 150 to about 1500 mg/day.

In still other embodiments, when the therapeutic agent comprises phensuximide, it is typical that the amount used is within a range of from about 300 to about 4000 mg/day, and even more typically, from about 600 to about 3500 mg/day.

Further, when the therapeutic agent comprises trimethadione, it is typical that the amount used is within a range of from about 300 to about 3000 mg/day, and even more typically, from about 600 to about 2700 mg/day. For children two to six years of age, it is typical that the amount used is within a range of from about 100 to about 2000 mg/day, and even more typically, from about 300 to about 1800 mg/day. For children up to two years of age, it is typical that the amount used is within a range of from about 50 to about 1600 mg/day, and even more typically, from about 150 to about 1200 mg/day.

When the therapeutic agent comprises paramethadione, it is typical that the amount used is within a range of from about 300 to about 3000 mg/day, and even more typically, from about 600 to about 2700 mg/day. For children two to six years of age, it is typical that the amount used is within a range of from about 100 to about 2000 mg/day, and even more typically, from about 300 to about 1800 mg/day. For children up to two years of age, it is typical that the amount used is within a range of from about 50 to about 1600 mg/day, and even more typically, from about 150 to about 1200 mg/day.

Alternatively, when the therapeutic agent comprises zonisamide, it is typical that the amount used is within a range of from about 10 to about 1200 mg/day, and even more typically from about 50 to about 750 mg/day.

In another alternative, when the therapeutic agent comprises flunarizine, it is typical that the amount used is within a range of about 1 to about 600 mg/day, and even more typically from about 5 to about 50 mg/day.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The timing of the administration of the therapeutic agent can also vary. For example, the agent can be administered beginning at a time prior to the hearing-loss event(s), during the hearing-loss event(s), or at a time after the hearing-loss event(s). Administration can be by a single dose, or more preferably a therapeutic agent of the present invention is given over an extended period. It is preferred that administration of the therapeutic agent begin some period before the hearing-loss event(s) and continue for the duration of the event(s). In certain embodiments, administration of the therapeutic agent is continued for 1 week, 2 weeks, 1 month, 3 months, 9 months, or one year after the hearing-loss event. In certain embodiments, administration of the therapeutic agent is continued throughout the life of the subject following the hearing-loss event.

Derivatives of succinimide, including ethosuximide, methsuximide, phensuximide, tetramethylsuccinimide, and α-methyl-α-phenylsuccinimide, derivatives of oxazolidiendione, including trimethadione and paramethadione, zonisamide, and flunarizine, are commercially available or may be made according to processes known in the art.

II. Indications To Be Treated or Prevented

The composition comprising a therapeutically effective amount of a succinimide derivative, a therapeutically effective amount of a oxazolidiendione derivative, a therapeutically effective amount of zonisamide, or a therapeutically effective amount of flunarizine may be employed to treat or prevent several non age related hearing impairments or non-age related disorders, such as damage to sensory hair cells or cochlear neurons. The therapeutic composition may be administered to either a subject that is at risk of developing any of the indications described herein or to treat a subject that already has any of the indications described herein.

In some aspects, the invention provides a method to treat damage to a sensory hair cell or a cochlear neuron due to any one or more of ototoxic drug exposure, sound trauma, surgical trauma (i.e., related to the surgical removal of a tumor on cranial nerve VIII), physical trauma (i.e., due to a fracture of the temporal bone affecting the inner and middle ear or due to a shearing injury affecting cranial nerve VIII), mercury, lead, toluene, disease, infection, and a genetic disorder. Typically, the severity of damage that may be treated will depend in large part on the nature and extent of an individual's exposure to any of the above-described stressors.

Ototoxic drugs include several types of antibiotics, such as aminoglycosides (i.e., gentamicin, erythromycin, streptomycin, tobramycin, neomycin, amikacin, netilmicin, etc.) and macrolide antibiotics (i.e., clarithromycin, azithromycin, roxithromycin), certain chemotherapeutic agents (i.e., actinomycin, bleomycin, cisplatin, carboplatin, nitrogen mustard, vincristine, dichloromethotrexate), certain diuretics, (i.e., furosemide (Lasix), bumetanide (Bumex), ethacrynic acid (Edecrin)), and NSAIDs as well as certain analgesics (i.e., Advil® and Motrin® (Ibuprofen), Aleve®, Naprosyn, Anaprox (Naproxen), Feldene, Dolobid, Indocin, Lodine, Relafin, Toradol, Volteran, Salicylates (aspirin, disalcid, Bufferin®, Ecotrin®, Trilisate, Ascriptin, Empirin, Excedrin®, Fiorinal).

In a further embodiment, the therapeutic agents are admistered to the subject to treat sound trauma. Sound trauma is a common source of hearing loss. In general, sound is characterized by its intensity (experienced as loudness) and frequency (experienced as pitch), and it is the intensity and duration of a noise exposure that determines the potential for harm to hair cells and cochlear neurons. Sound intensity is measured as sound pressure level (SPL) in a logarithmic decibel (dB) scale. The present invention may be utilized to treat sound having a variety of SPL and dB levels. Noise exposure is commonly measured in units of dB(A), a unit based on a scale weighted toward higher frequency sounds, to which the human ear is more sensitive. In certain embodiments, chronic sound exposure may be treated. Chronic exposures equal to an average SPL of 85 dB(A) or higher for an eight-hour period can cause permanent hearing loss. By way of example, a conversation exposes an individual to an SPL of 60 dB(A); a lawnmower exposes an individual to an SPL of 90 dB(A); and, stereo headphones expose an individual to an SPL of 110-120 dB(A). For more information regarding the SPLs of common types of noises and the risks of various noise exposures, see Noise-Induced Hearing Loss, by Peter M. Rabinowitz, M.D., M.P.H. (American Family Physician, May, 2000), which is hereby incorporated by reference. Examples of some common noise exposures include industrial/work related noise, i.e., jet takeoff, locomotive noise, recreational (non-work related) noise, gun shot noise, noise from chain saws and other power tools, amplified music, noise from recreational vehicles, such as snowmobiles, water craft, and motorcycles, and noise from some types of children's toys (Yaremchuk, et al., 1997). Industrial/work related noise typically causes a “noise notch,” with hearing loss occurring at mid-high frequencies bilaterally. Firearms, which are owned by some sixty million Americans, and other unilateral sources of noise cause more circumscribed lesions. The methods of the present invention can be used to treat damage to a sensory hair cell or a cochlear neuron due to any of the above-discussed noise exposures or any other noise exposure that has the potential to cause damage to the hair cell; the methods can be used before, during, and/or after the noise exposure.

In a further embodiment, the therapeutic agent may be administered to a subject to treat a disease associated with hearing loss. Generally speaking, diseases associated with hearing loss include most notably, Meniere's disease. Meniere's disease is associated with several symptoms, and not all sufferers exhibit the same symptoms. The four symptoms most commonly associated with Meniere's disease are vertigo or dizziness, fluctuating hearing loss, tinnitus, sensation of pressure in one or both ears. Meniere's disease frequently begins with one symptom, gradually progressing to include other symptoms; a diagnosis may be made in the absence of all four classic symptoms. Hearing loss associated with Meniere's disease may be unilateral (in one ear) or bilateral (in both ears) and commonly involves lower frequency sounds. Hearing loss may become progressively worse and may become permanent. Some individuals with unilateral hearing loss (by some accounts, as many as 50%) will develop bilateral hearing loss. Some individuals lose hearing entirely in one or both ears. Tinnitus can also worsen over time. The methods of the present invention can be used to treat damage to a sensory hair cell or a cochlear neuron due to Meniere's disease, at several stages of the disease, including before symptoms of hearing loss appear and after one or more symptoms of the disease have subsided. The methods of the present invention can also be used to treat those at risk for developing Meniere's disease. In addition, the methods of the present invention can be used to treat tinnitus.

Tinnitus, the perception of sound in the absence of acoustic stimulus (i.e., ringing, roaring, chirping, whooshing), is a stressful and sometimes incapacitating condition. The perceived sound can be intermittent or constant and its volume can vary from a quiet sound to a sound that drowns out all other sounds. Tinnitus may be objective, i.e., the sound can be detected by a physician, or subjective, i.e., the sound is only detected by the patient. Subjective tinnitus is most common. There is no cure for tinnitus; treatment usually involves treating the underlying cause of the tinnitus, i.e., Meniere's disease, head injury, stress, depression, or teaching patients coping techniques. For example, an individual suffering from chronic tinnitus may develop ways of masking the tinnitus sound with an artificial sound, i.e., from an electronic device. Tinnitus Retraining Therapy, which includes a combination of masking and psychological counseling, is commonly used with tinnitus patients. It is believed that there are two different types of subjective tinnitus, somatic tinnitus, which is linked to disorders within the head or neck but outside the ear, and otic tinnitus, which is linked to inner ear disorders, including disorders of the acoustic nerve. Other types of tinnitus include external ear tinnitus (which may involve the external ear canal or the ear drum), middle ear tinnitus (which may involve the middle ear chamber or the eustachian tube), inner ear tinnitus (which may involve the hair cells of the inner ear), nerve pathway tinnitus (which may involve cranial nerve VIII), and brain tinnitus (which may involve swelling of the brain). The methods of the present invention can be used to treat any type or degree of tinnitus, including subjective, objective, somatic, otic, external ear, middle ear, inner ear, nerve pathway, and brain tinnitus. The methods of the present invention may also be combined with any existent treatment for tinnitus, such as Tinnitus Retraining Therapy.

In an additional embodiment, the therapeutic agent is administered to treat hearing loss resulting from an infection. Several types of infections are associated with hearing loss, including bacterial and viral infections. Examples of such infections include labyrinthitis, syphilis, meningitis, mumps, and measles. Labyrinthitis refers to the inflammation of the inner ear or the nerves connecting the inner ear to the brain. Inflammation of the cochlea results in tinnitus and/or hearing loss. This inflammation can be the result of a bacterial or a viral infection. With regard to bacterial infections, bacteria and/or bacterial toxins can enter the inner ear as a result of bacterial meningitis or due to a rupture in the membranes that separate the middle ear from the inner ear (i.e., due to otitis media or perilymph fistula—a leakage of inner ear fluid to the middle ear associated with head trauma, physical exertion, or barotraumas). Viruses that cause inflammation in the inner ear are believed to enter the inner ear through the blood stream, e.g., via a local or systemic infection. Examples of common viruses that have been associated with labyrinthitis include influenza, measles (rubeola), mumps, German measles (rubella), herpes, hepatitis, polio, and Epstein-Barr. The methods of the present invention can be used to treat damage to a sensory hair cell or a cochlear neuron due to an infection, at any stage of the infection, including before symptoms of hearing loss appear and after one or more symptoms of the infection have subsided. The methods of the present invention can also be used to treat those at risk for acquiring an infection associated with hearing loss.

Hair cells and cochlear neurons may also be damaged or malfunction as a result of an underlying genetic disorder. By way of example, several genes have been identified as encoding key proteins associated with the stereocilia of hair cells, namely myosins VI, VIIA, and XV. Mutations in these genes impair transduction and have been shown to lead to deafness. A mutation in the murine gene encoding myosin VI results in progressive fusion of stereocilia; a mutation in the murine myosin XV gene results in short stereocilia; and a mutation in the murine myosin VIIA gene results in progressive disorganization of the stereocilia bundle. Mutations in myosins VIIA and XV have been associated with human deafness, also. Mutations in proteins that interact with one of these three myosins may also result in hearing impairment. For example, the protein harmonin, a protein present in stereocilia and known to underlie Usher syndrome type 1C (discussed in more detail below), may interact with myosin VIIA. The transmembrane protein vezatin, which binds to the myosin VIIA tail, is believed to be involved in stereocilia organization. Mutations in either one of these interacting proteins may be associated with hearing impairment. Additionally, mutated cadherin-related genes have been associated with the deaf mouse mutants waltzer and Ames waltzer, both of which show evidence of disorganization of the stereocilia bundle; the products of these genes may be involved in linking adjacent stereocilia. A frame shift mutation in the espin gene, which encodes the essential cytoskeletal component of stereocilia espin, has been associated with the deaf mouse mutant jerker. Other examples of genetic mutations associated with malfunctioning hair cells and resultant hearing impairment include mutations in the Atp2b2 gene and in the otoferlin gene (OTOF). The Atp2b2 gene is believed to encode a calcium pump in hair cells and is associated with the deaf waddler mouse mutant, a mutant that lacks a calcium pump. Mutations in the human OTOF gene have been reported in some cases of dominantly inherited, progressive deafness. Any of the above-described mutations can be detected using any one of a number of genetic testing methods known in the art. The methods of the present invention can be used to treat damaged or malfunctioning sensory hair cells or cochlear neurons associated with any of above-described genetic disorders.

The invention also provides methods that can be used to treat damage to a sensory hair cell or a cochlear neuron due to a combination of factors, such as a genetic disorder, ototoxic drug exposure, sound trauma, surgical trauma, physical trauma, mercury, lead, toluene, disease, and infection. For example, sound trauma is often a co-factor in hearing loss due to ototoxic drug exposure. Thus, those who suffer from hearing loss due to an ototoxic exposure may be at much greater risk for further hearing loss due to sound trauma. Those who consume salt in large quantities may also be more vulnerable to sound trauma.

In certain further aspects, the invention provides methods that can be used to treat non-age related hearing loss in a subject in need of such treatment. Particularly, in some aspects, the invention provides a method to treat non-age related hearing loss due to any one or more of ototoxic drug exposure, sound trauma, surgical trauma (i.e., related to the surgical removal of a tumor on cranial nerve VIII), physical trauma (i.e., due to a fracture of the temporal bone affecting the inner and middle ear or due to a shearing injury affecting cranial nerve VIII), mercury, lead, toluene, disease, infection, and a genetic disorder. Typically, the severity of damage that may be treated will depend in large part on the nature and extent of an individual's exposure to any of the above-described stressors. The above discussion of these stressors, i.e., types of ototoxic drugs, sound traumas, physical traumas, in the context of treating damage to sensory hair cells and cochlear neurons, is applicable in the context of treating non-age related hearing loss, as well. As with methods of treating damage to hair cells and cochlear neurons, methods of treating non-age related hearing loss associated with any of the above stressor exposures include administering the therapeutic agents of the invention, prior to, during, or after the stressor exposure, or to individuals at risk for the stressor exposure. Additionally, the invention provides methods of treating non-age related hearing loss due to a genetic disorder, including autosomal dominant, autosomal recessive, or X-linked disorders. Examples of genetic disorders in which hearing loss may be a symptom are Down syndrome (abnormality on a gene), Usher syndrome (type 1, 2, and 3) (autosomal recessive), Treacher Collins syndrome (autosomal dominant), Fetal alcohol syndrome (genetic abnormality), Crouzon syndrome (autosomal dominant), Alport syndrome (X-linked), Stickler syndrome (autosomal dominant), Waardenburg Syndrome, Pendred Syndrome, Norrie Syndrome, Branchial-oto-renal syndrome, and Jervell and Lange-Nielsen syndrome. The methods of the invention may be used to treat non-age related hearing loss associated with any of the above genetic disorders.

III. Diagnosis of Non-Age Related Hearing Loss

A further aspect of the invention encompasses diagnosing a subject in need of treatment or prevention for a hearing-loss event. A number of suitable methods for diagnosing hearing loss are known and may be used in the practice of the invention. Neurootological, neuroophthalmological, neurological examinations as well as electrooculography can be used in accordance with generally known methods. Sensitive and specific measures are available to identify patients with auditory impairments. For example, tuning fork tests can be used to differentiate a conductive hearing loss from a sensorineural hearing loss and to determine whether the loss is unilateral or bilateral. An audiometer is used to quantiate hearing loss, measured in decibels. With this device the hearing for each ear is measured, typically from 125 to 8000 Hz, and plotted as an audiogram. Speech audiometry can also be performed. The speech recognition threshold, the intensity at which speech is recognized as a meaningful symbol, can be determined at various speech frequencies. Speech or phoneme discrimination can also be determined and used as an indicator of sensorineural hearing loss, since analysis of speech sounds relies upon the inner ear and 8th nerve. Tympanometry can be used to diagnose conductive hearing loss and aid in the diagnosis of those patients with sensorineural hearing loss. Electrocochleography, measuring the cochlear microphonic response and action potential of the 8th nerve, and evoked response audiometry, measuring evoked response from the brainstem and auditory cortex to acoustic stimuli, can be used in patients, particularly in infants, children, and patients with sensorineural hearing loss of obscure etiology. These tests serve a diagnostic function as well as a clinical function in assessing potential response to therapy.

Sensory and neural hearing losses can be distinguished based on tests for recruitment (an abnormal increase in the perception of loudness or the ability to hear loud sounds normally despite a hearing loss), sensitivity to small increments in intensity, and pathologic adaptation, including stapedial reflex decay. Recruitment is generally absent in neural hearing loss. In sensory hearing loss the sensation of loudness in the affected ear increases more with each increment in intensity than it does in the normal ear. Sensitivity to small increments in intensity can be demonstrated by presenting a continuous tone of 20 db above the hearing threshold and increasing the intensity by 1 db briefly and intermittently. The percentage of small increments detected yields the “short increment sensitivity index” value. High values, 80 to 100%, are characteristic of sensory hearing loss, whereas a patient with a neural lesion and those with normal hearing cannot detect such small changes in intensity. Pathologic adaptation is demonstrated when a patient cannot continue to perceive a constant tone above the threshold of hearing; also known as tone decay. A Bekesy automatic audiometer or equivalent can be used to determine these clinical and diagnostic signs; audiogram patterns of the Type II, Type III, and Type IV variety are indicative of preferred types of hearing losses suitable for the treatment methods of the invention. As hearing loss can often be accompanied by vestibular impairment, vestibular function can be tested, particularly when presented with a sensorineural hearing loss of unknown etiology. When possible, diagnostics for hearing loss, such as audiometric tests, should be performed prior to exposure in order to obtain a patient normal hearing baseline. Upon exposure, particularly to an ototoxic drug, audiometric tests should be performed twice a week and testing should be continued even after cessation of the drug treatment since hearing loss may not occur until several days after cessation. U.S. Pat. No. 5,546,956 provides methods for testing hearing that may be used to diagnose a patient and monitor treatment. U.S. Pat. No. 4,637,402 provides a method of diagnosing a patient and monitoring treatment consisting of quantitatively measuring hearing loss. Both patents are hereby incorporated by reference in their entirety.

Definitions

The term “damage” as used herein in the context of sensory hair cells (including inner hair cells and outer hair cells), cochlear neurons, cochlear- and neuronal-supporting cells (including outer and inner pillar cells, inner and outer phalangeal cells, border cells, vestibular apparatus supporting cells, cells of Hensen, various neuroglial cells, Schwann cells, satellite cells, type II fibroblasts), includes defects in hair cells and neurons, i.e., genetic defects, as well as the malfunctioning of hair cells and neurons. The term “damage” also refers to the death of a sensory hair cell or a cochlear neuron, including apoptosis and necrosis.

As used herein, the term “death of sensory hair cells” refers to a cessation of the ability of one or more sensory hair cells in perceiving and/or transducing sensory stimuli.

As used herein, the term “effective amount,” refers to the amount of succinimide derivative, oxazolidiendione derivative, zonisamide, or flunarizine required to achieve an intended purpose for both prophylaxis and treatment, without undesirable side effects, such as toxicity, irritation or allergic response. Although individual needs may vary, the determination of optimal ranges for effective amounts of formulations is within the skill of the art.

By “exposure to an ototoxic agent” is meant that the ototoxic agent is made available to or comes into contact with a mammal. Exposure to an ototoxic agent can occur by direct administration, e.g., by ingestion or administration of a food, a medicinal agent, or a therapeutic agent, e.g., a chemotherapeutic agent, by accidental contamination, or by environmental exposure, e.g., aerial or aqueous exposure. Fetal exposure may also occur systemically when a drug given to the mother crosses the placental barrier.

The term “hearing impairment” refers to a defect in the ability to perceive sound and includes partial hearing loss, complete hearing loss, deafness (complete or partial), and tinnitus, the perception of non-existent sounds, i.e., a buzzing in the ear. The hearing impairment may be due to hair cell or neuron damage, wherein the damage is caused by a genetic disorder, loud sounds, ototoxicity, or any other such stressor described in the application. Hearing impairment includes sensorineural hearing loss, conductive hearing loss, combination hearing loss, mild (between 25 and 40 dB), moderate (between 41 and 55 dB), moderately severe (between 56 and 70 dB), severe (between 71 and 90 dB), and profound (90 dB or greater) hearing loss, congenital hearing loss, pre-lingual and post-lingual hearing loss, unilateral (affecting one ear) and bilateral (affecting both ears) hearing loss, or any combination of these, i.e., sensorineural/severe/postlingual/bilateral. The term does not include an impairment resulting from the natural aging process.

As used herein, the term “loss of sense of balance” refers to a deficit in the vestibular system of an animal compared to the vestibular system of a normally functioning subject.

“Mammal” refers to any animal classified as a mammal, including humans, domestic and farm animals, and zoo, sports, or pet animals, such as dogs, horses, cats, cows, etc. Preferably, the mammal herein is human.

As used herein, the term “sensory hair cells” refers to the hair cells present in vertebrates, including the auditory sensory hair cells present in the Organ of Corti, and the vestibular sensory hair cells present in the semicircular canals and maculae of the inner ear.

The term “subject in need of such treatment” or “patient in need of treatment” are used interchangeably herein, and refers to those already experiencing a hearing impairment, those prone to having a hearing impairment (e.g., those suffering from Meniere's disease), and/or those for whom exposure to one of the many stressors discussed in the application, i.e., sound trauma, ototoxic drugs, is predicted to occur (e.g., cancer patient about to begin chemotherapy with an ototoxic drug), is occurring, or has occurred.

The term “treat” or “treatment” as used herein in the context of hearing loss, loss of sense of balance, death of sensory hair cells or cochlear neurons, sensorineural hearing loss, or damage to sensory hair cells or cochlear neurons and the like, includes preventing the damage before it occurs, or reducing loss or damage or damage after it occurs. Both prophylactic and therapeutic measures is to prevent or diminish, respectively, hair cell- or neuron-damage-related hearing impairment, such as ototoxin-induced damage.

### EXAMPLES

The following examples illustrate the invention.

Methods

Animals and Procedures

All animal procedures were approved by the Animal Studies Committee at Washington University in St. Louis. A total of 44 C57BL/6J mice at 2-3 months old (The Jackson Laboratory, Bar Harbor, Me., USA) were used. In addition, a transgenic mouse line (YFP-12) expressing YFP under the thy-1 promoter (Feng et al., 2000) was crossed into C57BL/6J genetic background for over 10 generations. A total of 12 YFP-12 transgenic male mice at 2 months old were used. All mice were housed three to five per cage in a noise-controlled environment on a 12 hr light/dark cycle with light onset at 6:00 a.m.

Trimethadione and ethosuximide were obtained from Sigma Chemical Co. (St. Louis, Mo.). Based on our preliminary experiments, the dosage for trimethadione was 200 mg/day/kg (body weight); for ethosuximide 1.5 g/day/kg (body weight). These dosages were kept constant during the whole experimental duration based on the average amount of water uptake and body weight per cage, which were measured every three days. The drinking water with each drug were kept in dark bottles and changed every three days.

Animals in the group for testing potential prophylactic functions of trimethadione on NIHL were first tested by ABR and supplied with the respective drug in their drinking water for three weeks. They were then exposed to the 110 dB wide-band noise for 30 min. ABR testing followed 24 hours and 2-3 weeks after the noise exposure in order to estimate temporary and permanent threshold shift (TTS and PTS respectively).

Animals in the groups for testing potential therapeutic functions of trimethadione and ethosuximide were tested for ABR threshold first and exposed to the same noise the next day, and supplied with the respective drug right after the noise exposure in their drinking water for two weeks. ABR testing was performed 24 hours and 2-3 weeks after the noise exposure. All animals were sacrificed right after the last ABR testing with mixture of ketamine (80 mg/kg) and xylazine (16 mg/kg) given i.p.

Noise Exposure

Similar to approaches described previously (Ohlemiller et al.,

2000), noise exposures were performed in a foam-lined, double-walled soundproof room (Industrial Acoustics). The noise exposure apparatus consisted of a 21×21×11 cm wire cage mounted on a pedestal inserted into a B&K 3921 turntable. The cage was rotated at 1 revolution/80 s within a 42×42 cam metal bar frame. A Motorola KSN1020A piezo ceramic speaker (four total) was attached to each side of the frame. Opposing speakers were oriented nonconcentrically, parallel to the cage, and driven by independent channels of a Crown D150A power amplifier. Noise was generated by two General Radio 1310 generators and bandpassed at 4.0-45.0 kHz by Krohn-Hite 3550 filters. The overall noise level was measured at the center of the cage using a B&K 4135 ¼ inch microphone in a combination with a B&K 2231 sound level meter set a broadband (0.2 Hz-70 kHz). Mice were exposed in pairs at 110 dB SPL for 30 min.

ABR Recording

ABR assay was performed as described previously (Ohlemiller et al., 2000; Bao et al., 2005). The mouse cochlea typically responds to frequencies ranging from 2-100 kHz. The most sensitive region of the audiogram is roughly 5-40 kHz. To cover this range, we tested the frequencies of 5, 10, 20, 28.3 and 40 kHz (3 octaves). The “near field” sound stimulation and calibration was used in which the speaker was near the ear (7 cm) within the range where the sound field was approximately homogeneous within an imaginary cylinder surrounding the ear. To calibrate sound stimuli, a B&K 4135 ¼ inch microphone was placed where the mouse ear would normally be.

Prior to testing, all mice were anesthetized with 80 mg/kg ketamine and 15 mg/kg xylazine (i.p.). Otoscopic examination was performed to ensure that tympanic membranes were normal. Core temperature was maintained at 37+/−1° C. using a thermostatically-controlled heating pad in conjunction with a rectal probe (Yellow Springs Instruments Model 73A). Platinum needle electrodes (Grass) were inserted subcutaneously just behind the right ear (active), and at the vertex (reference), and in the back (ground). Electrodes were led to a Grass P15 differential amplifier (100-10,000 Hz, x100), then to a custom amplifier providing another x1,000 gain, finally digitized at 30 kHz using a Cambridge Electronic Design Micro1401 in conjunction with SIGNAL™ and custom signal averaging software operating on a 120 MHz Pentium PC. Sinewave stimuli generated by a HP 3445 oscillator were shaped by a custom electronic switch to 5 ms total duration, including 1 ms rise/fall times. The stimulus was amplified by a Crown D150A power amplifier and output to a KSN1020A piezo ceramic speaker. Toneburst stimuli at each frequency and level were presented 1,000 times at 20/sec. The minimum sound pressure level required for visual detection of a response (short-latency negative wave) was determined at selected frequencies, using a 5 dB minimum step size.

Histology

The methods for fixing, processing and analyzing the mouse cochlea have been described in detail elsewhere (Ohlemiller et al., 2000; Ou et al., 2000). Briefly, mice were perfused transcardially with cold 2% paraformaldehyde and 2% glutaraldehyde in 0.1 M phosphate buffer. Both cochleae were isolated, the stapes removed, and immersed in the same fixative. Complete exposure of the cochlea to the fixative was ensured by making a small hole at the apex of the cochlear capsule, gently circulating the fixative over the cochlea using a transfer pipet. After decalcification in sodium EDTA, the cochleae were post-fixed in buffered 1% osmium tetroxide, dehydrated in an ascending acetone series, and embedded in Epon. For SGN counting, mid-modiolar sections (4 μm) were cut and about fifty sections were collected for each cochlea. The number of SGN nuclei at the apex, middle, and base was counted every fifth section (total 10 sections per one cochlea) under a 200× visual field using a computerized planimetry program of Image Pro Plus (Media Cybernetics). Because all sections were cut in a similar orientation, it was possible to evaluate comparisons between two groups by Mann-Whitney tests. For hair cell counting, cochleae were dissected into roughly half-turn segments. These half-turn segments were examined in mineral oil under Nomarski optics, using a calibrated ocular. Three separate inner hair cell (IHC) and outer hair cell (OHC) counts were obtained from non-overlapping 100 μm segments. Cytocochleogrames were prepared for one pair of mice with the best prophylactic function of trimethadione. The percentages of missing IHCs and OHCs were determined by dividing the number of missing hair cells by the total number of hair cells (i.e. present plus absent) in the organ of Corti.

For the examination of SGN radial afferent terminals in YFP-12 transgenic mice, cochleae from YFP-12 mice were embedded in 25 mg/ml agrose (Sigma) and 7.7 mg/ml gelatin (Sigma). Flat cochlear sections were cut by a vibratome at 250 μm. For the examination of SGN terminals, cryostat midmodiolar sections at 40 μm were cut and collected. All these sections were processed for immunofluorescence staining of calretinin. Inhibition of non-specific binding and permeabilization steps were performed by incubating these sections for one hour with PBS containing 5% goat serum and 0.1% Triton X-100. Primary rabbit antibody against calretinin (Zymed, 1:200) and secondary antibodies (fluorescein- or cyanine 3-conjugated, Jackson Immunoresearch Laboratory) were incubated overnight and for two hours, respectively. Nuclei were stained with 1 μg/ml solution of Hoechst 33242 (Sigma). Coverslips were mounted with the antifading agent Vectashield (Vector Laboratories, USA). The sections were visualized using a Zeiss fluorescence microscope or a Leica TCS 4D confocal laser scanning microscope equipped with a krypton/argon mixed gas laser. For the comparison of YFP positive terminals in the cochlea among different experimental groups, the number of YFP terminals under hair cells were counted in every third midmodiolar section (40 μm) for total 10 sections after each section was imaged under a 600× visual field using a LaserSharp2000 program (Bio-Rad) and 3-D reconstructed a computer program (Volocity, Improvision Inc.).

### Example 1

### Testing of Trimethadione

Excessive noise is the greatest cause of permanent hearing loss although other factors such as aging and harmful chemicals also contribute (Henderson and Salvi, 1998; Ohlemiller et al., 2000; Davis et al., 2003; Harding et al., 2005). Immediately after noise exposure (within 24 hours), there is a temporary threshold shift (TTS). Two to three weeks after noise exposure, the still-elevated hearing threshold is called permanent threshold shift (PTS) (Clark, 1991; Quaranta et al., 1998; Nordmann et al., 2000). Previous studies have suggested that TTS and PTS are two distinct phenomena with different cellular pathological changes (Nordmann et al., 2000). A buckling of the pillar bodies (Nordmann et al., 2000), degeneration of type II fibrocytes of the spiral ligament and strial edema (Hirose and Liberman, 2003), and excitotoxic damage to afferent fibers (Pujol and Puel, 1999) may contribute to TTS. For PTS, histopathological correlations include: stereocilia damage, hair cell loss, and degeneration of afferent fibers in the organ of Corti. There are also massive losses of type II fibrocytes and degeneration of strial intermediate and marginal cells (Hirose and Liberman, 2003).

Experiments were designed to test whether succinimide derivatives have prophylactic activity for noise-induced hearing loss in inbred mice. Trimethadione was selected (TMO) as the first candidate for testing because (1) TMO is approved for human use to treat absence seizures, and extensive toxicological studies have been done; and (2) TMO is water soluble and can be precisely measured for oral dosage. Mice were dosed with TMO at 200 mg/kg body weight/day in their drinking water for three weeks before the noise exposure. Their body weight and water uptake were monitored every three days. The TMO solution was maintained in colored bottles and changed every three days.

To examine the possible effects of TMO on noise-induced hearing loss, the ABR functional analysis was used to measure hearing thresholds in mice before and after noise exposure. (K. K Ohlemiler, J. S. Wright, A. F. Hedibreeder, Hear Res 149:239-247 (2000)) Briefly, animals are anesthetized, for example with i.p. ketamine and xylazine, and positioned in a headholder. The animal's core temperature is maintained using a heating pad with thermal probe feedback control. Subcutaneous platinum needle electrodes are inserted proximal to an ear, and are connected to suitable amplifying and data collection equipment, which are used to record auditory evoked potentials. Auditory sine wave stimuli are presented using an oscillator, suitable amplifiers, and a piezo ceramic speaker located proximally to the ear. Systematic presentation of auditory stimuli at various frequencies in increasing volume steps of a least 5 dB permits collection of auditory evoked potentials that are used to evaluate auditory threshold in the animal. The results from control animals and animals that have received treatment with a compound being tested are compared to evaluate whether the test compound delays hearing loss.

Before the noise exposure, there was no difference in ABR threshold between the control (n=5, 8 month-old) and the TMO-treated group (n=5, 8 month-old), one week after TMO treatment (FIG. 1A). Twenty-four hours after noise exposure (wideband, 110 dB for 30 minutes), the temporary threshold shift (TTS) was significantly lower at all seven frequencies tested in the TMO-treated mice compared to the control mice (FIG. 1B). A similar observation was also made for the permanent threshold shift (PTS) 14 days after the same noise exposure (FIG. 1C). Thus, TMO can delay both TTS and PTS after noise exposure.

### Example 2

### Testing of Ethosuximide

Next Zarontin (i.e., ethosuximide), a similar succinimide derivative, was tested for a therapeutic function after noise-exposure. Immediately after the noise exposure, mice were dosed with ethosuximde in their drinking water for three weeks at 400 mg/kg body weight/day. Similar to the results obtained from the TMO study, before the noise exposure, there was no difference in ABR threshold between the control (n=5, 2 month-old) and the ethosuximide-treated group (n=5, 2 month-old) (FIG. 2A). Twenty-four hours after noise exposure (wideband, 110 dB for 30 minutes), the TTS was significantly lower in the ethosuximide-treated mice compared to the control mice (FIG. 2B). A similar observation was also made for the PTS 21 days after the same noise exposure (FIG. 2C). Thus, ethosuximide has therapeutic functions to both TTS and PTS after noise exposure.

### Example 3

### Therapeutic Function of Trimethadione and Ethosuximide

There was no significant difference in pre-exposure ABR thresholds (Pre-Noise-Only and Pre-Noise-Drug, FIGS. 3A and 3B) for either male or female mice. For TTS (Noise-Only-24h and Noise-Drug-24h), male mice fed with trimethadione in their drinking water right after the noise exposure showed about 10 dB less ABR threshold shift than controls across all five frequencies we tested (FIG. 3A; P<0.05). A trend of reduced ABR threshold shift was also observed in female mice after fed with trimethadione although there was no significant difference in female mice between the control and trimethadione-treated mice (FIG. 3B). A similar pattern was found for the PTS (two weeks after the noise exposure; Noise-Only-2w and Noise-Drug-2w). The ABR shift was significantly less in male mice treated with trimethadione than control male mice (P<0.01; FIG. 1A), while there was no statistical difference for female mice between the control and trimethadione treated group (FIG. 3B). Since this was the first time that trimethadione, a blocker for T-type calcium channels, was found able to treat NIHL in male mice, another blocker for T-type calcium channels, ethosuximide, was used to confirm this finding. As predicted, male mice fed with ethosuximide in their drinking water right after the noise exposure showed less ABR threshold shift than the control (FIG. 4; P<0.05 for the TTS and P<0.01 for PTS).

### Example 4

### Prophylactic Function of Trimethadione

In both male and female mice, trimethadione dramatically reduced the ABR threshold shift after the noise exposure. In male mice, trimethadione prevented about 4.3 dB shift for TTS and 9.7 dB shift for PTS at five tested frequencies from 5 to 40K (Drug-Noise-24h and Drug-Noise-2w; FIG. 5A). At every frequency tested, trimethadione-treated mice had much less ABR threshold shift than the control mice (P<0.01 for both TTS and PTS). In female mice, trimethadione prevented about 5.0 dB shift for TTS and 7.0 dB shift for PTS across five tested frequencies (Drug-Noise-24h and Drug-Noise-2w; P<0.01 for both TTS and PTS; FIG. 5B).

### Example 5

### Histological Findings

No obvious gross morphological changes were observed in mid-modiolar sections between the control and drug-treated mice, which were under the treatment of either trimethadione or ethosuximide. Quantification data showed a trend for the loss of more outer hair cells at the basal region of the cochleae in control mice than in drug-treated mice (Noise-Only-2w and Noise-Drug-2w; FIG. 6A), while there was no difference in the number of SGNs between the control and drug-treated mice (FIG. 6B). Similarly, there was no difference in the SGN number between the control and mice treated with ethosuximide despite of its significant therapeutic effect (FIG. 7).

Among the groups testing for the prophylactic function of trimethadione, no obvious morphological differences were observed. A pair of 3-moth-old mice from these groups was selected for constructing detailed cytocochleograms (FIG. 8). In this pair of mice, there was about 35 dB of permanent ABR threshold shift among five frequencies tested in the control mouse, and only 11 dB shift in the mouse receiving trimethadione two weeks before the noise exposure. At the base region (32 kHz to 64 kHz), there were about 22.2% missing IHCs and 74.4% OHCs in the control mouse, while there were about 1.8% missing IHCs and 68.8% OHCs in the drug-treated mouse. Before the noise exposure, the ABR threshold at 40 kHz for both mice was 28 dB. Thus, for this specific pair, there were more hair cells in the drug-treated mouse after the noise exposure. However, even in this pair, quantitatively morphological observations were not well correlated with ABR assays. For example, at the apex region (0 to 8 kHz), there were about 25 dB shifts for the control mouse and no shift for the drug-treated mouse, while there was about 0.3% and 4.6% loss of IHCs and OHCs respectively in the control mouse, and 1.9% and 3.5% loss of IHCs and OHCs in the drug-treated mouse.

Since noise-induced acoustic damages may occur in different targets in the cochlea, and with our noise exposure procedure, no consistent changes were found in the number of IHCs, OHCs, or SGNs, we examined the neuronal connections between hair cells and SGNs by taking advantage of a transgenic Y12 mouse line, which we recently found a strong YFP expression in SGNs and their terminals (FIG. 9). We first examined SGN radial afferent fibers in the surface preparations. No consistent changes were found between control and noise-exposed cochleae although a subtle dis-organization of these fibers was noted in noise-exposed groups (FIG. 9A; left two panels). Interestingly, a decease of YFP-positive terminals underneath IHCs and OHCs was observed, and some of them underneath OHCs originate from the tunnel crossing efferent (FIG. 9A; right two panels). These YFP terminals were quantified at the middle region in every third midmodiolar section (40 μm). Consistent with initial examinations, a trend of reduced YFP terminals underneath OHCs was found in mice two weeks after noise exposure although there was no statistical difference between two noise-exposed groups and the control group (P=0.353). The YFP terminals underneath IHCs were significantly reduced underneath IHCs two weeks after noise exposure (P<0.001 between the control and the noise-only group, and P=0.002 between the control and the drug-noise group). The number of YFP terminals was slightly higher in the drug-noise group compared to the noise-only group (underneath OHCs, 16.3+/−6.0 vs. 14.9+/−7.4; underneath IHCs, 11.0+/−2.8 vs. 10.3+/−2.9) although there was no statistical difference between these two groups.

### REFERENCES

Bao, J., Lei, D., Du, Y., Ohlemiller, K. K., Beaudet, A L., Role, L. W., 2005. Requirement of nicotinic acetylcholine receptor subunit beta2 in the maintenance of spiral ganglion neurons during aging. J. Neurosci., 25 3041-3045.
Boettcher, F. A., 1996. Diltiazem does not protect the ear from noise-induced hearing loss in mongolian gerbils. Laryngoscope., 106 772-776.
Boettcher, F. A., Caldwell, R. K., Gratton, M. A., White, D. R., Miles, L. R., 1998. Effects of nimodipine on noise-induced hearing loss. Hear. Res., 121 139-146.
Clark, W. W., 1991. Recent studies of temporary threshold shift (TTS) and permanent threshold shift (PTS) in animals. J. Acoust. Soc. Am., 90 155-163.
Davis, R. R., Kozel, P., Erway, L. C., 2003. Genetic influences in individual susceptibility to noise: a review. Noise Health., 5 19-28.
Errington, A. C., Stohr, T., Lees, G., 2005. Voltage gated ion channels: targets for anticonvulsant drugs. Curr. Top. Med. Chem., 5 15-30.
Farooqui, A. A., Ong, W. Y., Horrocks, L. A., 2004. Biochemical aspects of neurodegeneration in human brain: involvement of neural membrane phospholipids and phospholipases A2. Neurochem. Res., 29 1961-1977.
Feng G, Mellor R H, Bernstein M, Keller-Peck C, Nguyen Q T, Wallace M, Nerbonne J M, Lichtman J W, Sanes J R., 2000. Imaging neuronal subsets in transgenic mice expressing multiple spectral variants of GFP. Neuron. 28 41-51.
Fuchs, P. A., 1996. Synaptic transmission at vertebrate hair cells. Curr. Opin. Neurobiol., 6 514-519.
Harding, G. W., Bohne, B. A., Vos, J. D., 2005. The effect of an age-related hearing loss gene (Ahl) on noise-induced hearing loss and cochlear damage from low frequency noise. Hear. Res., 204 90-100.
Heinrich U R, Maurer J, Mann W., 1997. Alteration of loosely bound calcium in the guinea pig organ of Corti after treatment with diltiazem as calcium channel blocker. Eur. Arch. Otorhinolaryngol., 254 223-229.
Henderson, D., Salvi, R. J., 1998. Effects on noise exposure on the auditory functions. Scand. Audiol. Suppl., 48 63-73.
Hirose, K., Liberman, M. C., 2003. Lateral wall histopathology and endocochlear potential in the noise-damaged mouse cochlea. J. Assoc. Res. Otolaryngol., 4 339-352.
Hunter-Duvar, I. M., Elliott, D. N., 1972. Effects of intense auditory stimulation: hearing losses and inner ear changes in the squirrel monkey. J. Acoust. Soc. Am., 52 1181-1192.
Ison, J. R., Payman, G. H., Palmer, M. J., Walton, J. P., 1997. Nimodipine at a dose that slows ABR latencies does not protect the ear against noise. Hear. Res., 106 179-183.
Kochegarov, A. A., 2003. Pharmacological modulators of voltage-gated calcium channels and their therapeutically application. Cell Calcium., 33 145-162.
Kopke, R., Bielefeld, E., Liu, J., Zheng, J., Jackson, R., Henderson, D., Coleman, J. K., 2005. Prevention of impulse noise-induced hearing loss with antioxidants. Acta. Otolaryngol., 125 235-243.
Kujawa S G, Liberman M C., 2006. Acceleration of age-related hearing loss by early noise exposure: evidence of a misspent youth. J Neurosci. 26 2115-2123.
Lacinova, L., Klugbauer, N., Hofmann, F., 2000. Low voltage activated calcium channels: from genes to function. Gen. Physiol. Biophys., 19 121-136.
Layton, M. G., Robertson, D., Everett, A. W., Mulders, W. H., Yates, G. K., 2005. Cellular localization of voltage-gated calcium channels and synaptic vesicle-associated proteins in the guinea pig cochlea. J. Mol. Neurosci., 27 225-244.
Le Prell, C. G., Dolan, D. F., Schacht, J., Miller, J. M., Lomax, M. I., Altschuler, R. A., 2003. Pathways for protection from noise induced hearing loss. Noise Health., 5 1-17.
Lopez, I., Ishiyama, G., Acuna, D., Ishiyama, A., Baloh, R. W., 2003. Immunolocalization of voltage-gated calcium channel alpha1 subunits in the chinchilla cochlea. Cell Tissue Res., 313 177-186.
Luer, M. S., Rhoney, D. H., Hughes, M., Hatton, J., 1996. New pharmacologic strategies for acute neuronal injury. Pharmacotherapy., 16 830-848.
Lynch, E. D., Kil, J., 2005. Compounds for the prevention and treatment of noise-induced hearing loss. Drug Discov. Today., 10 1291-1298.
Mills, J. H., Matthews. L. J., Lee, F. S., Dubno, J. R., Schulte, B. A., Weber, P. C., 1999. Gender-specific effects of drugs on hearing levels of older persons. Ann. N Y Acad. Sci., 884 381-388.
Minami, S. B., Yamashita, D., Schacht, J., Miller, J. M., 2004. Calcineurin activation contributes to noise-induced hearing loss. J. Neurosci. Res., 78 383-392.
Morley, B. J., Li, H. S., Hiel, H., Drescher, D. G., Elgoyhen, A. B., 1998. Identification of the subunits of the nicotinic cholinergic receptors in the rat cochlea using RT-PCR and in situ hybridization. Brain Res. Mol. Brain Res., 53 78-87.
Niedzielski, A. S., Safieddine, S., Wenthold, R. J., 1997. Molecular analysis of excitatory amino acid receptor expression in the cochlea. Audiol. Neurootol., 2 79-91.
Niu, X., Canlon, B., 2002. Protective mechanisms of sound conditioning. Adv. Otorhinolaryngol., 59 96-105.
Nordmann, A. S., Bohne, B. A., Harding, G. W., 2000, Histopathological differences between temporary and permanent threshold shift. Hear. Res., 139 13-30.
Ohlemiller, K. K., 2006. Contributions of mouse models to understanding of age- and noise-related hearing loss. Brain Res. 1091 89-102.
Ohlemiller, K. K., Wright, J. S., Heidbreder, A. F., 2000. Vulnerability to noise-induced hearing loss in ‘middle-aged’ and young adult mice: a dose-response approach in CBA, C57BL, and BALB inbred strains. Hear. Res., 149 239-247.
Ou, H. C., Bohne, B. A., Harding, G. W., 2000. Noise damage in the C57BL/CBA mouse cochlea. Hear. Res., 145 111-122.
Parks, T. N., 2000. The AMPA receptors of auditory neurons. Hear. Res., 147 77-91.
Perez-Reyes, E., 1998. Molecular characterization of a novel family of low voltage-activated, T-type, calcium channels. J. Bioenerg. Biomembr., 30 313-318.
Pujol, R., Puel, J. L., 1999. Excitotoxicity, synaptic repair, and functional recovery in the mammalian cochlea: a review of recent findings. Ann. N Y Acad. Sci., 884 249-254.
Quaranta, A., Portalatini, P., Henderson, D., 1998. Temporary and permanent threshold shift: an overview. Scand. Audiol. Suppl., 48 75-86.
Robertson D., 1983. Functional significance of dendritic swelling after loud sounds in the guinea pig cochlea. Hear Res. 9(3) 263-78.
Seixas, N. S., Goldman, B., Sheppard, L., Neitzel, R., Norton, S., Kujawa, S. G., 2005. Prospective noise induced changes to hearing among construction industry apprentices. Occup. Environ. Med., 62 309-317.
Snutch, T. P., Reiner, P. B., 1992. Ca2+ channels: diversity of form and function. Curr. Opin. Neurobiol., 2 247-253.
So, H. S., Park, C., Kim, H. J., Lee, J. H., Park, S. Y., Lee, J. H., Lee, Z. W., Kim, H. M., Kalinec, F., Lim, D. J., Park, R., 2005. Protective effect of T-type calcium channel blocker flunarizine on cisplatin-induced death of auditory cells. Hear. Res., 204 127-139.
Sullivan, P. G., Springer, J. E., Hall, E. D., Scheff, S. W., 2004. Mitochondrial uncoupling as a therapeutic target following neuronal injury. J. Bioenerg. Biomembr., 36 353-356.
Triggle, D. J., 2006. L-type calcium channels. Curr. Pharm. Des., 12 443-157.
Toescu, E. C., Verkhratsky, A., Landfield, P. W., 2004. Ca2+ regulation and gene expression in normal brain aging. Trends Neurosci., 27 614-620.
Wang, Y., Liberman, M. C., 2002. Restraint stress and protection from acoustic injury in mice. Hear. Res., 165 96-102.
Yoon Y., 2005. Regulation of mitochondrial dynamics: another process modulated by Ca2+ signals? Sci. STKE., 2005 pe18.
Yunker, A. M., McEnery, M. W., 2003. Low-voltage-activated (“T-Type”) calcium channels in review. J. Bioenerg. Biomembr., 35 533-575.
Zipfel, G. J., Babcock, D. J., Lee, J. M., Choi, D. W., 2000. Neuronal apoptosis after CNS injury: the roles of glutamate and calcium. J. Neurotrauma., 17 857-869.

All references cited in the preceding text of the patent application or in the following reference list, to the extent that they provide exemplary, procedural, or other details supplementary to those set forth herein, are specifically incorporated by reference to the same extent as if each individual publication or patent application was specifically and individually indicated to be incorporated by reference.

What is claimed is:

1. A method for treating a non-age related hearing impairment in a subject in need of such treatment, the method comprising administering a derivative of a succinimide compound to the subject.

2. The method of claim 1, wherein the succinimide derivative is ethosuximide.

3. The method of claim 1, wherein the succinimide derivative is selected from the group consisting of methsuximide, phensuximide, tetramethylsucinimide, and α-methyl-α-phenylsuccinimide.

4. The method of claim 1, further comprising diagnosing the subject as having a non-age related hearing impairment or for being at risk for developing a non-age related hearing impairment.

5. The method of claim 1, wherein the non-age related hearing impairment is selected from a group of conditions consisting of tinnitus, sensorineural hearing loss, neural hearing loss, and Meniere's disease.

6. The method of claim 1, further comprising administering a therapeutically effective amount of the succinimide derivative, the therapeutically effective amount comprising a dose from about 50 to about 1000 mg/kg of body weight of the subject per day.

7. A method for treating a non-age related hearing impairment in a subject in need of such treatment, the method comprising administering a derivative of an oxazolidiendione compound to the subject.

8. The method of claim 7, wherein the oxazolidiendione derivative is trimethadione.

9. The method of claim 7, wherein the oxazolidiendione derivative is paramethadione.

10. The method of claim 7, further comprising diagnosing the subject as having a non-age related hearing impairment or for being at risk for developing a non-age related hearing impairment.

11. The method of claim 7, wherein the non-age related hearing impairment is selected from a group of conditions consisting of tinnitus, sensorineural hearing loss, neural hearing loss, and Meniere's disease.

12. The method of claim 7, further comprising administering a therapeutically effective amount of the oxazolidiendione derivative, the therapeutically effective amount comprising a dose from about 50 to about 1000 mg/kg of body weight of the subject per day.

13. A method for treating damage to sensory hair cells or damage to cochlear neurons in a subject in need of such treatment, the method comprising administering a derivative of a succinimide compound to the subject.

14. The method of claim 13, wherein the succinimide derivative is ethosuximide.

15. The method of claim 13, wherein the succinimide derivative is selected from the group consisting of methsuximide, phensuximide, tetramethylsucinimide, and α-methyl-α-phenylsuccinimide.

16. The method of claim 13, further comprising diagnosing the subject as having damage to the sensory hair cells or to the cochlear neurons or for being at risk for developing damage to the sensory hair cells or to the cochlear neurons.

17. The method of claim 13, further comprising administering a therapeutically effective amount of the succinimide derivative, the therapeutically effective amount comprising a dose from about 50 to about 1000 mg/kg of body weight of the subject per day.

18. A method for treating damage to sensory hair cells or damage to cochlear neurons in a subject in need of such treatment, the method comprising administering a derivative of an oxazolidiendione compound to the subject.

19. The method of claim 18, wherein the oxazolidiendione derivative is trimethadione.

20. The method of claim 18, wherein the oxazolidiendione derivative is paramethadione.

21. The method of claim 18, further comprising diagnosing the subject as having damage to the sensory hair cells or to the cochlear neurons or for being at risk for developing damage to the sensory hair cells or to the cochlear neurons.

22. The method of claim 18, further comprising administering a therapeutically effective amount of the succinimide derivative, the therapeutically effective amount comprising a dose from about 50 to about 1000 mg/kg of body weight of the subject per day.
